(19) United States
(12) Patent Application Publication
(10) Pub. No.: US 2018/0263461 A1
(43) Pub. Date: Sep. 20, 2018

(54) MEDICAL CONTROL APPARATUS, CONTROL METHOD, AND PROGRAM

(71) Applicant: SONY CORPORATION, TOKYO (JP)

(72) Inventors: TOSHIKI ITO, KANAGAWA (JP); TETSUYA MITANI, TOKYO (JP); CHIKAKO KIDO, KANAGAWA (JP); KEIICHI YOSHIOKA, TOKYO (JP)

(21) Appl. No.: 15/761,346

(22) PCT Filed: Aug. 10, 2016
(86) PCT No.: PCT/JP2016/073515

§ 371 Date: Mar. 19, 2018

(30) Foreign Application Priority Data
Oct. 2, 2015 (JP) 2015-196416

(51) Int. Cl. A61B 1/00

(52) U.S. Cl. CPC A61B 1/00006; A61B 1/00039; A61B 1/00045; A61B 1/00016

(57) ABSTRACT

Provided is a medical control apparatus including: a processing unit configured to restrict a function which is executable at a control target apparatus in a case where an operation is being performed, on a basis of whether or not an operation is being performed.

### TECHNICAL FIELD

The present disclosure relates to a medical control apparatus, a control method, and a program.

### BACKGROUND ART

A technology relating to a medical control system in which a plurality of apparatuses are connected through communication has been developed. Examples of the technology relating to the medical control system can include, for example, a technology disclosed in the following Patent Literature 1 and a technology disclosed in the following Patent Literature 2.

### CITATION LIST

### Patent Literature

Patent Literature 1: JP 2001-000449A

Patent Literature 2: JP 2006-000536A

### DISCLOSURE OF INVENTION

### Technical Problem

A medical control system includes a medical control apparatus such as an operating room (OR) controller to perform, for example, control of input/output of an image (a still image or a moving image), control relating to new entry and change of information relating to a patient on which an operation is to be performed, or the like. Here, an existing medical control apparatus can always perform all types of control.

However, because the existing medical control apparatus can always perform all types of control, for example, in the case where a user such as a healthcare professional performs erroneous manipulation with respect to an operation screen relating to control during an operation, control in response to the erroneous manipulation is performed. Therefore, in the case where the user performs erroneous manipulation during an operation when the existing medical control apparatus is used, there is a possibility of inhibiting progression of the operation.

The present disclosure proposes new and improved medical control apparatus, control method and program which can realize prevention of user erroneous manipulation during an operation.

### Solution to Problem

According to the present disclosure, there is provided a medical control apparatus including: a processing unit configured to restrict a function which is executable at a control target apparatus in a case where an operation is being performed, on a basis of whether or not an operation is being performed.

Further according to the present disclosure, there is provided a control method to be executed by a medical control apparatus, the control method including: a step of restricting a function which is executable at a control target apparatus in a case where an operation is being performed, on a basis of whether or not an operation is being performed.

Further according to the present disclosure, there is provided a program for causing a computer to implement a function of restricting a function which is executable at a control target apparatus in a case where an operation is being performed, on a basis of whether or not an operation is being performed.

### Advantageous Effects of Invention

According to the present disclosure, it is possible to realize prevention of user erroneous manipulation during an operation.

Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1 is an explanatory diagram illustrating an example of a medical control system according to the present embodiment.

FIG. 2 is an explanatory diagram for explaining an example of display at the medical control system to which a control method according to the present embodiment is applied.

FIG. 3 is an explanatory diagram for explaining an example of display at the medical control system to which a control method according to the present embodiment is applied.

FIG. 4 is an explanatory diagram for explaining an example of display at the medical control system to which a control method according to the present embodiment is applied.

FIG. 5 is an explanatory diagram for explaining an example of display at the medical control system to which a control method according to the present embodiment is applied.

FIG. 6 is an explanatory diagram for explaining an example of display at the medical control system to which a control method according to the present embodiment is applied.

FIG. 7 is an explanatory diagram for explaining an example of display at the medical control system to which a control method according to the present embodiment is applied.

FIG. 8 is an explanatory diagram for explaining an example of display at the medical control system to which a control method according to the present embodiment is applied.

FIG. 9 is an explanatory diagram for explaining an example of display at the medical control system to which a control method according to the present embodiment is applied.

FIG. 10 is an explanatory diagram for explaining an example of display at the medical control system to which a control method according to the present embodiment is applied.

FIG. 11 is an explanatory diagram for explaining an example of display at the medical control system to which a control method according to the present embodiment is applied.

FIG. 12 is an explanatory diagram for explaining an example of display at the medical control system to which a control method according to the present embodiment is applied.

FIG. 13 is an explanatory diagram for explaining an example of display at the medical control system to which a control method according to the present embodiment is applied.

FIG. 14 is an explanatory diagram for explaining an example of display at the medical control system to which a control method according to the present embodiment is applied.

FIG. 15 is an explanatory diagram for explaining an example of display at the medical control system to which a control method according to the present embodiment is applied.

FIG. 16 is an explanatory diagram for explaining an example of display at the medical control system to which a control method according to the present embodiment is applied.

FIG. 17 is an explanatory diagram for explaining an example of display at the medical control system to which a control method according to the present embodiment is applied.

FIG. 18 is an explanatory diagram for explaining an example of display at the medical control system to which a control method according to the present embodiment is applied.

FIG. 19A is an explanatory diagram for explaining an example of display at the medical control system to which a control method according to the present embodiment is applied.

FIG. 19B is an explanatory diagram for explaining an example of display at the medical control system to which a control method according to the present embodiment is applied.

FIG. 20 is a block diagram illustrating an example of a configuration of a medical control apparatus according to the present embodiment.

FIG. 21 is an explanatory diagram illustrating an example of a hardware configuration of the medical control apparatus according to the present embodiment.

### MODE(S) FOR CARRYING OUT THE INVENTION

Hereinafter, a preferred embodiment of the present disclosure will be described in detail with reference to the appended drawings. Note that, in this specification and the appended drawings, structural elements that have substantially the same function and structure are denoted with the same reference numerals, and repeated explanation of these structural elements is omitted.

Further, in the following description, description will be provided in the following order.
1. Control method according to present embodiment
2. Medical control apparatus according to present embodiment
3. Program according to present embodiment

### (Control Method According to Present Embodiment)

First, a control method according to the present embodiment will be described. In the following description, an example will be described where a medical control apparatus according to the present embodiment performs processing associated with the control method according to the present embodiment.

[1] Processing Associated with Control Method According to Present Embodiment

As described above, because the existing medical control apparatus can always perform all types of control, in the case where the user performs erroneous manipulation during an operation when the existing medical control apparatus is used, there is a possibility of inhibiting progression of the operation.

Therefore, the medical control apparatus according to the present embodiment restricts functions which can be executed at a control target apparatus on the basis of whether or not an operation is being performed (function control processing).

Here, the control target apparatus according to the present embodiment is an apparatus for which functions to be executed are to be controlled by the medical control apparatus according to the present embodiment. Examples of the control target apparatus can include, for example, an arbitrary apparatus such as a tablet type apparatus, a computer such as a personal computer (PC) and a communication apparatus such as a smartphone.

In the medical control system including the medical control apparatus according to the present embodiment, there may be one control target apparatus, or a plurality of control target apparatuses. In the case where there are a plurality of control target apparatuses, the medical control apparatus according to the present embodiment can, for example, restrict functions of each of the control target apparatuses by performing function control processing, or can change functions to be restricted for each of the control target apparatuses. Further, the medical control apparatus according to the present embodiment can, for example, also change functions to be restricted for each user who is utilizing the control target apparatus by performing function control processing regardless of the number of control target apparatuses included in the medical control system.

The medical control apparatus according to the present embodiment discerns whether or not an operation is being performed, for example, by a state of an “object whose state is switched between a state where an operation is being performed and a state where an operation is not being performed” displayed on a display screen.

Here, examples of the “object whose state is switched between a state where an operation is being performed and a state where an operation is not being performed” can include, for example, an Intra-OP button, or the like, which will be described later. In the following description, there is a case where the “object whose state is switched between a state where an operation is being performed and a state where an operation is not being performed” will be referred to as an “operation state switching object”.

Further, the medical control apparatus according to the present embodiment may discern whether or not an operation is being performed, for example, on the basis of an operation signal indicating whether or not an operation is being performed, acquired from an operation device. Here, examples of the operation device relating to the operation signal indicating whether or not an operation is being performed can include, for example, a physical button which can switch between a state where an operation is being performed and a state where an operation is not being performed, a remote controller, or the like.

Then, in the case where an operation is being performed, the medical control apparatus according to the present embodiment restricts functions which can be executed at the control target apparatus. Specific examples of a method for restricting functions which can be executed at the control target apparatus according to the present embodiment will be described later.

Here, examples of the functions which can be executed at the control target apparatus, to be restricted by the medical control apparatus according to the present embodiment can include, for example, one or more of the following functions.
Function of setting information relating to a patient on which an operation is to be performed (for example, a function relating to editing of information relating to a patient, such as newly registration of information relating to a patient and change of information relating to a patient)
Log-out function
Function of putting application into a sleep state
Function of stopping processing being executed at the control target apparatus (for example, stopping recording of image data, or the like)

By the above-described function of setting information relating to a patient on which an operation is to be performed being restricted, information relating to the patient cannot be newly registered or changed at the control target apparatus. Further, the above-described log-out function being restricted, log-out cannot be performed at the control target apparatus. Still further, by the above-described function of putting the application into a sleep state being restricted, the application is not put into a sleep state at the control target apparatus. Further, by the above-described function of stopping processing being executed at the control target apparatus being restricted, for example, recording of image data cannot be stopped, or the like, at the control target apparatus.

Note that, it goes without saying that the functions which can be executed at the control target apparatus, to be restricted by the medical control apparatus according to the present embodiment are not limited to the above-described examples. For example, the medical control apparatus according to the present embodiment can also restrict manipulation itself with respect to the operation state switching object. An example of restricting manipulation with respect to the operation state switching object will be described later.

Here, in the case where the functions to be restricted, which can be executed at the control target apparatus, include a function of setting information relating to a patient on which an operation is to be performed, change of information relating to the patient is prevented during the operation.

Because it is often the case that information relating to a patient on which an operation is to be performed (for example, arbitrary items relating to the patient, such as name, age, sex and a medical state) is settled at a time point when the operation is performed, it is normally not assumed that the information relating to the patient is changed during the operation. Therefore, manipulation of changing the information relating to the patient during the operation by a user such as a healthcare professional who is involved in the operation is highly likely to be user erroneous manipulation.

Therefore, as a result of the functions to be restricted, which can be executed at the control target apparatus including a function of setting information relating to a patient on which an operation is to be performed, it is, for example, possible to prevent manipulation of changing the information relating to the patient during the operation by a user such as a healthcare professional who is involved in the operation, as user erroneous manipulation.

The medical control apparatus according to the present embodiment, for example, restricts functions set in advance among the functions which can be executed at the control target apparatus.

Note that the functions to be restricted by the medical control apparatus according to the present embodiment, which can be executed at the control target apparatus are not limited to the above-described examples.

For example, the medical control apparatus according to the present embodiment can change functions to be restricted at the control target apparatus as described in the following (A) and (B).

(A) First Example of Changing Functions to be Restricted at Control Target Apparatus: Restriction in Accordance with User Authority

The medical control apparatus according to the present embodiment restricts functions which can be executed at the control target apparatus on the basis of user authority information corresponding to the user who is utilizing the control target apparatus.

Here, examples of the user who is utilizing the control target apparatus according to the present embodiment can include, for example, a user who is logging in to the control target apparatus.

Further, the user authority information according to the present embodiment is, for example, data including user identification information indicating the user and function identification information indicating a function. Examples of the user authority information can include, for example, a table (or a database) in which a user ID (an example of the user identification information) is associated with one or more function IDs (an example of the function identification information). For example, user authority information including a user ID which matches a user ID allocated to a certain user corresponds to user authority information corresponding to the user.

The medical control apparatus according to the present embodiment, for example, acquires a user ID of a user who is logging in to the control target apparatus from the control target apparatus. The medical control apparatus according to the present embodiment then specifies functions to be restricted, corresponding to the user who is logging in to the control target apparatus on the basis of the acquired user ID and the user authority information. The medical control apparatus according to the present embodiment then restricts the specified functions at the control target apparatus.

By the functions being restricted on the basis of the user authority information as described above, it is possible to realize restriction of the functions in accordance with the user authority. Further, by the functions being restricted on the basis of the user authority information, it is also possible to realize that, for example, “functions are not restricted even during an operation at a control target apparatus to which a specific user is logging in”. Therefore, by the medical control apparatus according to the present embodiment restricting the functions on the basis of the user authority information as described above, it is possible to realize a medical control system which can restrict functions more flexibly.

(B) Second Example of Changing Functions to be Restricted at Control Target Apparatus: Restriction in Accordance with Authority of Control Target Apparatus

The medical control apparatus according to the present embodiment restricts functions which can be executed at the control target apparatus on the basis of apparatus authority information corresponding to the control target apparatus.

Here, the apparatus authority information according to the present embodiment is, for example, data including apparatus identification information indicating an apparatus and function identification information. Examples of the apparatus authority information can include, for example, a table (or a database) in which an apparatus ID (an example of the apparatus identification information) is associated with one or more function IDs (an example of the function identification information). For example, apparatus authority information including an apparatus ID which matches an apparatus ID allocated to a certain control target apparatus corresponds to apparatus authority information corresponding to the control target apparatus.

The medical control apparatus according to the present embodiment, for example, acquires an apparatus ID from the control target apparatus. The medical control apparatus according to the present embodiment then specifies functions to be restricted corresponding to the control target apparatus on the basis of the acquired apparatus ID and apparatus authority information. The medical control apparatus according to the present embodiment then restricts the specified functions at the control target apparatus.

By the functions being restricted on the basis of the apparatus authority information as described above, it is possible to realize restriction of functions in accordance with authority of the control target apparatus. Further, by the functions being restricted on the basis of the apparatus authority information, it is, for example, also possible to realize that “functions are not restricted even during an operation at a specific control target apparatus such as an administration terminal”. Therefore, by the medical control apparatus according to the present embodiment restricting the functions on the basis of the apparatus authority information as described above, it is possible to realize a medical control system which can restrict functions more flexibly.

The medical control apparatus according to the present embodiment can change functions to be restricted at the control target apparatus for each user who is logging in to the control target apparatus (a user who is utilizing the control target apparatus) or for each control target apparatus, for example, as described in the above (A) and (B).

Specifically, the medical control apparatus according to the present embodiment restricts functions which can be executed at the control target apparatus as described in the following (1) to (3).

### (1) First Example of Function Control Processing

The medical control apparatus according to the present embodiment causes the operation state switching object to be displayed on a display screen. The medical control apparatus according to the present embodiment causes the operation state switching object to be displayed on an arbitrary operation screen which is displayed on the display screen and which allows the user to perform manipulation, such as the device selection screen and the device control screen which will be described later.

Examples of the display screen according to the present embodiment can include, for example, a display screen of a display device provided at the control target apparatus and a display screen of a display device connected to the control target apparatus. Further, the display device constituting the display screen according to the present embodiment may be a device which can perform display and which allows the user to perform manipulation, such as, for example, a touch panel.

Here, examples of the user manipulation with respect to the operation screen can include, for example, the following examples. Note that, it goes without saying that the examples of the user manipulation with respect to the operation screen are not limited to the following examples.
Manipulation of touching the operation screen (in the case where the display device is a touch panel)
Manipulation using an operation device such as a physical button provided at the control target apparatus or an external operation device of the control target apparatus, such as a remote controller

Then, in the case where the operation state switching object indicates a state where an operation is being performed, the medical control apparatus according to the present embodiment restricts functions which can be executed at the control target apparatus.

Further, as described above, the medical control apparatus according to the present embodiment can change the functions to be restricted at the control target apparatus for each user who is utilizing the control target apparatus or for each control target apparatus.

Further, in the case where the state indicated by the operation state switching object becomes a state where an operation is not being performed while functions which can be executed at the control target apparatus are limited, the medical control apparatus according to the present embodiment withdraws restriction of the functions.

Note that the function control processing according to the first example is not limited to the above-described examples.

For example, the medical control apparatus according to the present embodiment can change whether or not to display the operation state switching object for each user who is utilizing the control target apparatus or for each control target apparatus as described in the following (a) and (b).

(a)

The medical control apparatus according to the present embodiment, for example, causes the operation state switching object to be selectively displayed on the display screen on the basis of user authority information corresponding to the user who is utilizing the control target apparatus.

Specifically, for example, in the case where it is specified in the user authority information corresponding to the user who is utilizing the control target apparatus that the operation state switching object is caused to be displayed, the medical control apparatus according to the present embodiment causes an operation screen in which the operation state switching object is displayed to be displayed on the display screen. Meanwhile, for example, in the case where it is not specified in the user authority information corresponding to the user who is utilizing the control target apparatus that the operation state switching object is caused to be displayed, the medical control apparatus according to the present embodiment does not cause the operation screen in which the operation state switching object is displayed to be displayed on the display screen.

In the case where the operation state switching object is not caused to be displayed on the display screen, the medical control apparatus according to the present embodiment does not restrict functions which can be executed at the control target apparatus.

Meanwhile, in the case where the operation state switching object is caused to be displayed on the display screen, the medical control apparatus according to the present embodiment restricts functions which can be executed at the control target apparatus.

As described above, in the case where the functions which can be executed at the control target apparatus are restricted, the medical control apparatus according to the present embodiment can change functions to be restricted at the control target apparatus for each user who is utilizing the control target apparatus.

As an example, the medical control apparatus according to the present embodiment restricts manipulation with respect to the operation state switching object on the basis of the user authority information corresponding to the user who is utilizing the control target apparatus. For example, in the case where a function of performing manipulation with respect to the operation state switching object is specified in the user authority information corresponding to the user who is utilizing the control target apparatus, the medical control apparatus according to the present embodiment restricts the manipulation with respect to the operation state switching object at the control target apparatus.

(b)

The medical control apparatus according to the present embodiment, for example, causes the operation state switching object to be selectively displayed on the display screen on the basis of the apparatus authority information corresponding to the control target apparatus.

Specifically, for example, in the case where it is specified in the apparatus authority information corresponding to the control target apparatus that the operation state switching object is caused to be displayed, the medical control apparatus according to the present embodiment causes the operation screen in which the operation state switching object is displayed to be displayed on the display screen. Meanwhile, for example, in the case where it is not specified in the apparatus authority information corresponding to the control target apparatus that the operation state switching object is caused to be displayed, the medical control apparatus according to the present embodiment does not cause the operation screen in which the operation state switching object is displayed to be displayed on the display screen.

In the case where the operation state switching object is not caused to be displayed on the display screen, the medical control apparatus according to the present embodiment does not restrict functions which can be executed at the control target apparatus.

Meanwhile, in the case where the operation state switching object is caused to be displayed on the display screen, the medical control apparatus according to the present embodiment restricts functions which can be executed at the control target apparatus.

As described above, in the case where functions which can be executed at the control target apparatus are restricted, the medical control apparatus according to the present embodiment can change functions to be restricted at the control target apparatus for each control target apparatus.

As an example, the medical control apparatus according to the present embodiment restricts manipulation with respect to the operation state switching object on the basis of the apparatus authority information corresponding to the control target apparatus. For example, in the case where a function of performing manipulation with respect to the operation state switching object is specified in the apparatus authority information corresponding to the control target apparatus, the medical control apparatus according to the present embodiment restricts manipulation with respect to the operation state switching object at the control target apparatus.

### (2) Second Example of Function Control Processing

The medical control apparatus according to the present embodiment restricts functions which can be executed at the control target apparatus on the basis of an operation signal which indicates whether or not an operation is being performed, acquired from an operation device such as a physical button and a remote controller.

In the case where an operation signal indicating that an operation is being performed is acquired, the medical control apparatus according to the present embodiment restricts functions which can be executed at the control target apparatus. As described above, the medical control apparatus according to the present embodiment can change functions to be restricted at the control target apparatus for each user who is utilizing the control target apparatus or each control target apparatus.

Further, in the case where an operation signal indicating that an operation is not being performed is acquired while functions which can be executed at the control target apparatus are restricted, the medical control apparatus according to the present embodiment withdraws restriction of the functions.

### (3) Third Example of Function Control Processing

The medical control apparatus according to the present embodiment can also perform both the function control processing according to the first example described in the above (1) and the function control processing according to the second example described in the above (2).

The medical control apparatus according to the present embodiment performs the function control processing, for example, as described above as the processing associated with the control method according to the present embodiment.

Here, the medical control apparatus according to the present embodiment restricts functions which can be executed at the control target apparatus in the case where an operation is being performed on the basis of whether or not an operation is being performed.

Therefore, for example, because it is possible to minimize manipulation which can be performed by the user during an operation, the medical control apparatus according to the present embodiment can prevent user erroneous manipulation during an operation. Further, as a result of erroneous manipulation being prevented and unnecessary manipulation being unable to be performed during an operation by functions being restricted, it is possible to make work of the user during an operation more efficient.

Note that the processing associated with the control method according to the present embodiment is not limited to the above-described function control processing.

For example, the medical control apparatus according to the present embodiment can further perform notification processing of one or both of notification processing described in the following (i) and notification processing described in the following (ii).

### (i) First Example of Notification Processing

The medical control apparatus according to the present embodiment, for example, notifies a predetermined user that an operation is being performed or an operation is finished on the basis of a state of the operation state switching object such as an Intra-OP switch which will be described later. Further, the medical control apparatus according to the present embodiment can, for example, also notify a predetermined user that an operation is being performed or an operation is finished on the basis of the operation signal acquired from the operation device.

The medical control apparatus according to the present embodiment, for example, transmits an e-mail indicating that an operation is being performed or an e-mail indicating that an operation is finished to an e-mail address of a user registered in advance (an example of the predetermined user) by executing e-mail application. Further, the medical control apparatus according to the present embodiment may, for example, transmit a message indicating that an operation is being performed or a message indicating that an operation is finished to an address of the user registered in advance by executing message application. Note that the medical control apparatus according to the present embodiment can notify the predetermined user that an operation is being performed or an operation is finished using an arbitrary method which is capable of notifying the predetermined user such as a registered user.

By the notification processing according to the first example being performed, it is, for example, possible to make the predetermined user recognize that an operation is being performed or an operation is finished.

### (ii) Second Example of Notification Processing

For example, in the case where functions of the control target apparatus are restricted, the medical control apparatus according to the present embodiment causes a notification that functions of the control target apparatus are restricted to be made.

The medical control apparatus according to the present embodiment causes a notification that functions of the control target apparatus are restricted to be made using a visual notification method such as, for example, “causing a message indicating that functions are restricted to be displayed on the display screen in the case where manipulation associated with the restricted functions is performed” and “causing a warning lamp indicating that functions are restricted to be lighted in the case where manipulation associated with the restricted functions is performed”.

Further, the medical control apparatus according to the present embodiment may cause a notification that functions of the control target apparatus are restricted to be made using an auditory notification method such as, for example, “causing a sound output device such as a speaker to output sound (including music) indicating that functions are restricted in the case where manipulation associated with the restricted functions is performed”.

Further, the medical control apparatus according to the present embodiment can also cause a notification that functions of the control target apparatus are restricted to be made using an arbitrary notification method which can notify the user that functions of the control target apparatus are restricted, such as, for example, combination of the visual notification method as described above and the auditory notification method as described above.

By the notification processing according to the second example being performed, it is, for example, possible to make a user such as a healthcare professional who is involved in the operation, and who is using the device selection screen or the device control screen which will be described later, recognize that functions of the control target apparatus are restricted.

Note that each of the “above-described function control processing” and the “above-described function control processing and the above-described notification processing” is processing divided from the processing associated with the control method according to the present embodiment for convenience sake. Therefore, in the processing associated with the control method according to the present embodiment, for example, the “above-described function control processing and the above-described notification processing” can be regarded as one type of processing. Further, in the processing associated with the control method according to the present embodiment, for example, each of the “above-described function control processing” and the “above-described function control processing and the above-described notification processing” can be regarded as two or more types of processing (in accordance with an arbitrary way of dividing the processing).

### [2] Example of Display in Medical Control System to Which Control Method According to Present Embodiment is Applied

The control processing associated with the control method according to the present embodiment will be described next using an example of display in the medical control system to which the control method according to the present embodiment is applied.

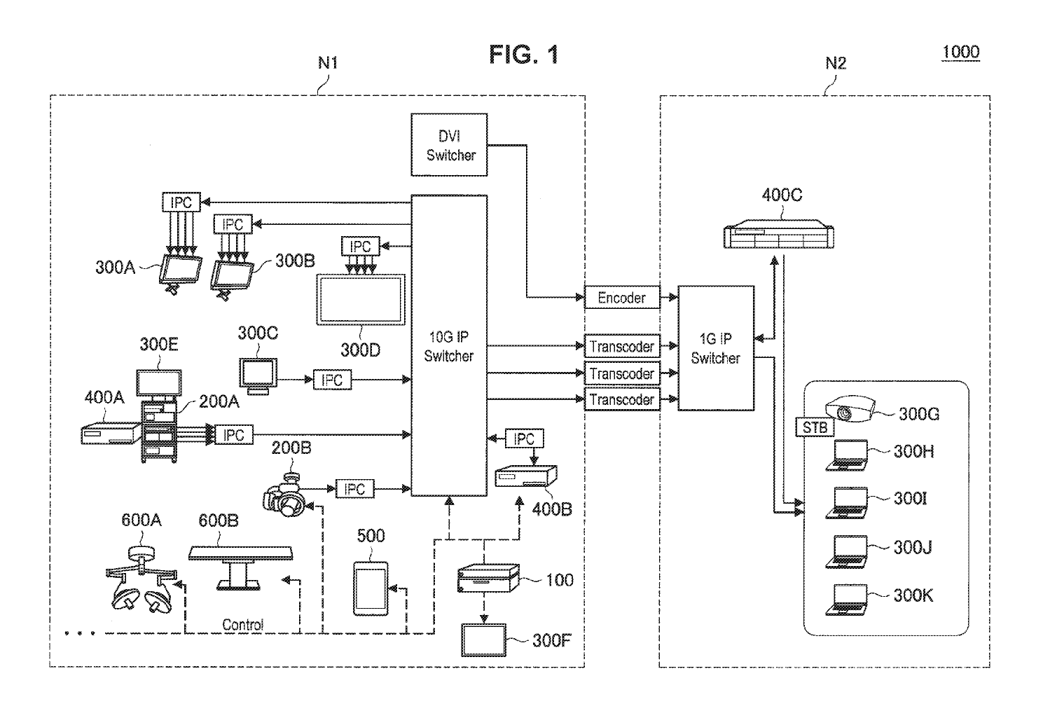

FIG. 1 is an explanatory diagram illustrating an example of the medical control system 1000 according to the present embodiment.

FIG. 1 illustrates an example of a system in which an image signal which is made to conform to IP is transmitted among apparatuses via an internet protocol converter (IPC), a switcher, an encoder, a transcoder, or the like. In FIG. 1, for example, a “10G IP Switcher”, a “DVI Switcher” and a “1G IP Switcher” correspond to the switchers. Further, in FIG. 1, for example, an “Encoder” corresponds to the encoder, and, for example, a “Transcoder” corresponds to the transcoder. Note that, in the medical control system according to the present embodiment, it is also possible to employ a configuration where an image signal which is made to conform to IP is not transmitted.

Further, FIG. 1 illustrates an example where the medical control system 1000 includes a network N1 inside the operating room and a network N2 outside the operating room. Note that the medical control system 1000 may include, for example, only the network N1 inside the operating room.

The medical control system 1000 includes, for example, a medical control apparatus 100, input source apparatuses 200A, 200B, . . . , output destination apparatuses 300A, 300B, . . . , apparatuses 400A, 400B, . . . , having functions of one or both of the input source and the output destination, a display target apparatus 500 at which various kinds of control screens are to be displayed, and other apparatuses 600A, 600B, . . . , controlled by the medical control apparatus 100. The respective apparatuses are connected, for example, through wired communication of an arbitrary communication scheme or through wireless communication of an arbitrary communication scheme via an apparatus such as the IPC and the switcher.

Examples of the input source apparatuses 200A, 200B, . . . , can include an arbitrary apparatus which becomes a signal source of an image signal, such as medical equipment having an imaging function such as an endoscope (for example, the input source apparatus 200A) and an imaging device provided in the operating room, or the like, (for example, the input source apparatus 200B), for example, as illustrated in FIG. 1. In the following description, there is a case where the input source apparatuses 200A, 200B, . . . , will be collectively referred to as an “input source apparatus 200” or one of the input source apparatuses 200A, 200B, . . . , will be referred to as the “input source apparatus 200”.

Examples of the output destination apparatuses 300A, 300B, . . . , can include, for example, an arbitrary apparatus which can become an output destination of an image, such as a display device which can display an image. Examples of the display device can include, for example, a monitor provided in the operating room (for example, the output destination apparatuses 300A to 300F), an image projection apparatus such as a projector (for example, the output destination apparatus 300G), a monitor provided at the PC, or the like, (for example, the output destination apparatuses 300H to 300K) as illustrated in FIG. 1. In the following description, there is a case where the output destination apparatuses 300A, 300B, . . . , will be collectively referred to as an “output destination apparatus 300” or one of the output destination apparatuses 300A, 300B, . . . , will be referred to as the “output destination apparatus 300”.

Examples of the apparatuses 400A, 400B, . . . , having functions of one or both of the input source and the output destination can include an apparatus having functions of one or both of a function of recording an image in a recording medium and a function of reproducing image data stored in the recording medium. Specifically, examples of the apparatuses 400A, 400B, . . . , having functions of one or both of the input source and the output destination can include a recorder (for example, the apparatuses 400A and 400B) and a server (for example, the apparatus 400C), for example, as illustrated in FIG. 1. In the following description, there is a case where the apparatuses 400A, 400B, . . . , having functions of one or both of the input source and the output destination will be collectively referred to as an “apparatus 400” or one of the apparatuses 400A, 400B, . . . , having functions of one or both of the input source and the output destination will be referred to as the “apparatus 400”. Further, in the following description, an example where the apparatus 400 is a recorder will be mainly described.

The display target apparatus 500 is an apparatus at which the device selection screen and the device control screen which will be described later are to be displayed. The display target apparatus 500 corresponds to an example of the control target apparatus according to the present embodiment.

Examples of the display target apparatus 500 can include, for example, an arbitrary apparatus such as a tablet type apparatus, a computer such as a PC, a communication apparatus such as a smartphone. In FIG. 1, a tablet type apparatus including a touch panel is illustrated as the display target apparatus 500. Note that the medical control system 1000 may include a plurality of display target apparatuses. Further, in the medical control system 1000, for example, one of the medical control apparatus 100, the input source apparatus 200, the output destination apparatus 300 and the apparatus 400 may play a role of the display target apparatus.

Examples of other apparatuses 600A, 600B, . . . , can include a lighting apparatus at an operating table (other apparatus 600A), the operating table (other apparatus 600B), or the like, for example, as illustrated in FIG. 1. Note that the medical control apparatus 100 does not have to have a function of controlling the other apparatuses 600A, 600B, . . . .

(I) A device selection screen and (II) a device control screen will be described below as an example of display to be displayed on a display screen of the display target apparatus 500 in the medical control system 1000 as illustrated in FIG. 1. Note that, for example, in the case where the medical control system 1000 includes a plurality of display target apparatuses (control target apparatuses), content displayed in (I) the device selection screen and (II) the device control screen is shared among the plurality of display target apparatuses by being controlled by the medical control apparatus 100.

### (I) Device Selection Screen

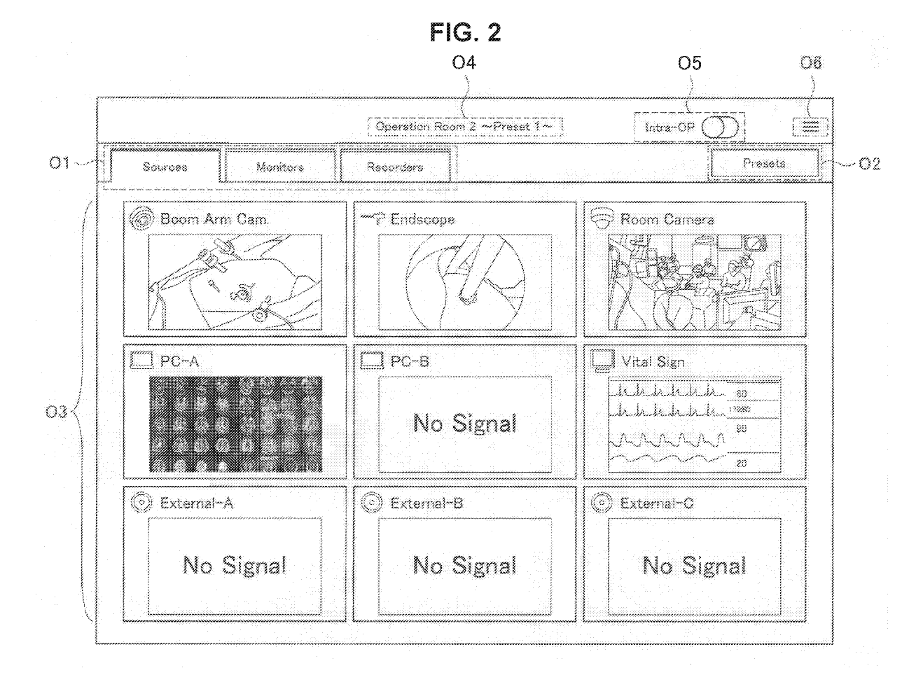

FIG. 2 to FIG. 9 are explanatory diagrams for explaining an example of display in the medical control system 1000 to which the control method according to the present embodiment is applied. FIG. 2 to FIG. 9 each illustrates the whole device selection screen or part of the device selection screen to be displayed on the display screen of the display target apparatus 500. The device selection screen will be described below while referring to FIG. 2 to FIG. 9 as appropriate.

FIG. 2 illustrates an example of the device selection screen. Referring to FIG. 2, the device selection screen includes, for example, a device category selection tab (O1 illustrated in FIG. 2), a preset button (O2 illustrated in FIG. 2), a device selection button (O3 illustrated in FIG. 2), location name display (O4 illustrated in FIG. 2), an Intra-OP switch (O5 illustrated in FIG. 2), and a context menu button (O6 illustrated in FIG. 2).

The device category selection tab (O1 illustrated in FIG. 2) is a tab for switching a category of a device which can be displayed on a display screen. In FIG. 2, as the category of the device which can be displayed on the display screen, “Sources” corresponding to the input source apparatus 200, “Monitors” corresponding to the output destination apparatus 300 and “Recorders” corresponding to the apparatus 400 are illustrated. Here, FIG. 2 illustrates a case where “Sources” is selected in the device category selection tab.

The preset button (O2 illustrated in FIG. 2) is a button for making the screen transition to a screen set in advance. Here, the preset button may be highlighted, for example, by being colored, by being blinked, or the like.

The device selection button (O3 illustrated in FIG. 2) is a button for selecting a device, and by the device selection button being manipulated, the screen transitions to the device control screen which will be described later. Because, in FIG. 2, “Sources” is selected in the device category selection tab, by the device selection button being manipulated, the screen transitions to a Sources screen in the device control screen which will be described later.

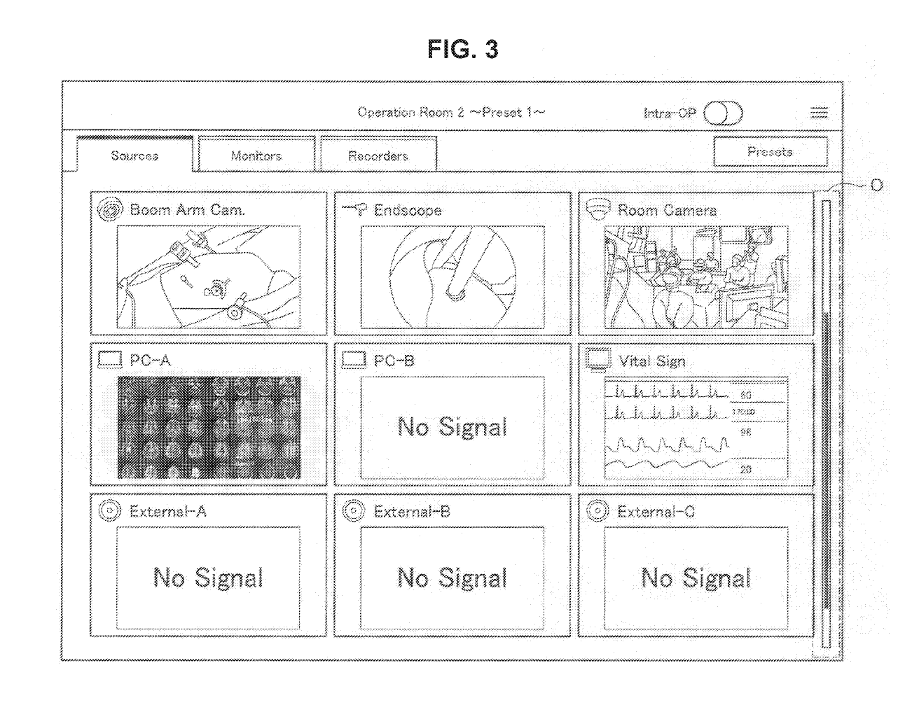

Here, in the case where there are too many device selection buttons to be displayed within one screen when each of the device selection buttons is displayed at a size illustrated in FIG. 2, a scroll bar is displayed, for example, as indicated with O in FIG. 3.

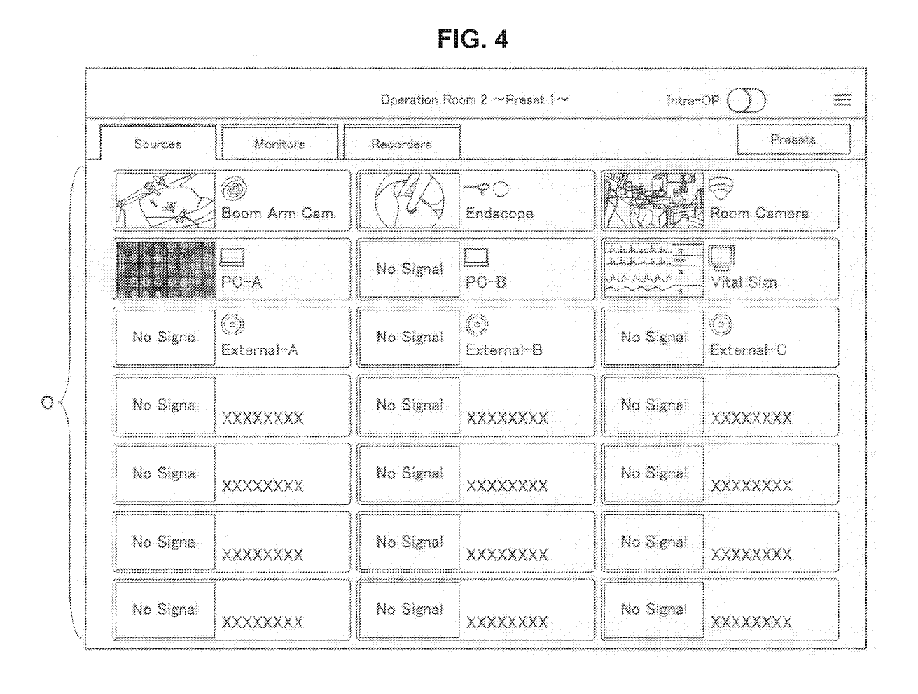

Further, in the case where there are too many device selection buttons to be displayed within one screen when each of the device selection buttons is displayed at a size illustrated in FIG. 2, each of the device selection buttons may be displayed at a smaller size, for example, as illustrated in FIG. 4.

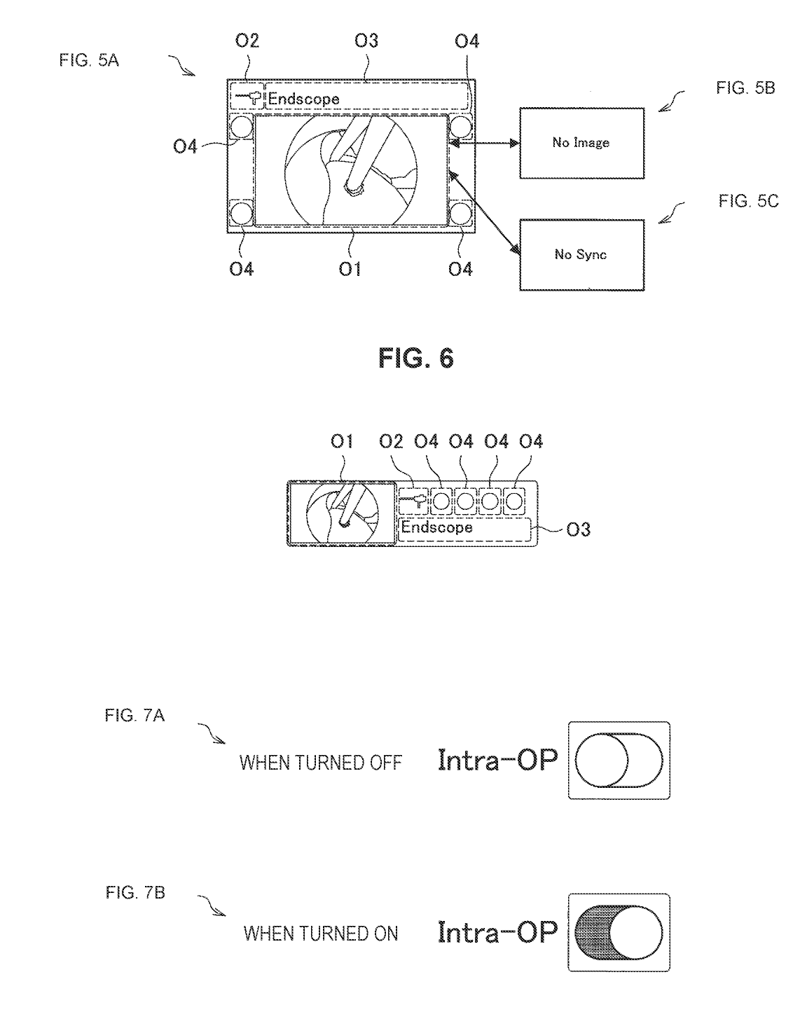

FIG. 5 illustrates an example of a configuration of the device selection button, and illustrates an example of the device selection button displayed in the device selection screen illustrated in FIG. 2.

The device selection button displayed in the device selection screen illustrated in FIG. 2 includes, for example, a thumbnail image (O1 illustrated in FIG. 5), an icon (O2 illustrated in FIG. 5), device name display (O3 illustrated in FIG. 5) and status display (O4 illustrated in FIG. 5) as illustrated in A of FIG. 5.

The thumbnail image (O1 illustrated in FIG. 5) illustrated in FIG. 5 indicates an image output by a device which is an input source.

Here, while examples of the thumbnail image can include a still image, the thumbnail image may be a moving image. As described above, the thumbnail image according to the present embodiment can be regularly or irregularly updated. In the case where the thumbnail image is updated, it is possible to reflect an image most recently output from the input source in the thumbnail image.

Further, for example, in the case where a parameter of a device which is an input source is adjusted, or the like, in the device control screen which will be described later, the image output from the input source after the parameter is adjusted is reflected in the thumbnail image. As an example, in the case where a parameter associated with zoom of the device which is the input source is adjusted in the device control screen which will be described later, the zoomed image is reflected in the thumbnail image.

Further, in the case where the thumbnail image cannot be acquired, display as illustrated in B of FIG. 5 or C of FIG. 5 is performed in accordance with the following states.
In the case where a thumbnail has not been acquired yet: B in FIG. 5
In the case where there is no input yet: C in FIG. 5

The icon (O2 illustrated in FIG. 5) is an icon indicating a type of the device.

The device name display (O3 in FIG. 5) indicates name of the device. A region of the device name display (O3 in FIG. 5) is set so as to stretch over the width of the device selection button so that, for example, long device name can be displayed.

The status display (O4 illustrated in FIG. 5) indicates a state of the device with simple display like an icon. The status display is displayed at four corners, for example, as illustrated in A in FIG. 5. Further, in the case where the status display is displayed at four corners, up to four types of status display can be displayed.

FIG. 6 illustrates another example of the configuration of the device selection button and illustrates an example of a configuration of the device selection button displayed in the device selection screen illustrated in FIG. 4.

The device selection button displayed in the device selection screen illustrated in FIG. 4 includes a thumbnail image (O1 illustrated in FIG. 6), an icon (O2 illustrated in FIG. 6), device name display (O3 illustrated in FIG. 6) and status display (O4 illustrated in FIG. 6), for example, in a similar manner to the device selection button illustrated in FIG. 4, as illustrated in A of FIG. 6.

Referring to FIG. 2 again, an example of the device selection screen will be described. The location name display (O4 illustrated in FIG. 2) indicates a location such as operating room name which is currently being selected.

The Intra-OP switch (O5 illustrated in FIG. 2) is a switch for switching between a state where an operation is being performed and a state where an operation is not being performed. The Intra-OP switch (O5 illustrated in FIG. 2) corresponds to an example of the operation state switching object according to the present embodiment. Here, in the case where the Intra-OP switch is in an ON state which indicates that an operation is being performed, by the function control processing according to the present embodiment being performed, user manipulation set in advance, editing of various kinds of data, or the like, are restricted in the device selection screen and the device control screen which will be described later.

FIG. 7 illustrates an example of a state of the Intra-OP switch. In the case where the Intra-OP switch is put into an OFF state which does not indicate that an operation is being performed, the Intra-OP switch is, for example, put into a state as illustrated in A of FIG. 7. In this event, the display target apparatus 500 is put into a state where restriction of functions which can be executed at the display target apparatus 500 by the function control processing according to the present embodiment is withdrawn. Further, in the case where the Intra-OP switch is put into an ON state which indicates that an operation is being performed, the Intra-OP switch is, for example, put into a state as illustrated in B of FIG. 7, and is more highlighted than in a case of the OFF state.

As illustrated in B of FIG. 7, by highlighting the Intra-OP switch more in a case of the ON state than the Intra-OP switch in a case of the OFF state, it is, for example, possible to allow the user to recognize that the state is a state indicating that an operation is being performed.

Referring to FIG. 2 again, an example of the device selection screen will be described. The context menu button (O6 illustrated in FIG. 2) is a menu button for allowing the user to perform detailed manipulation, editing of various kinds of data, or the like.

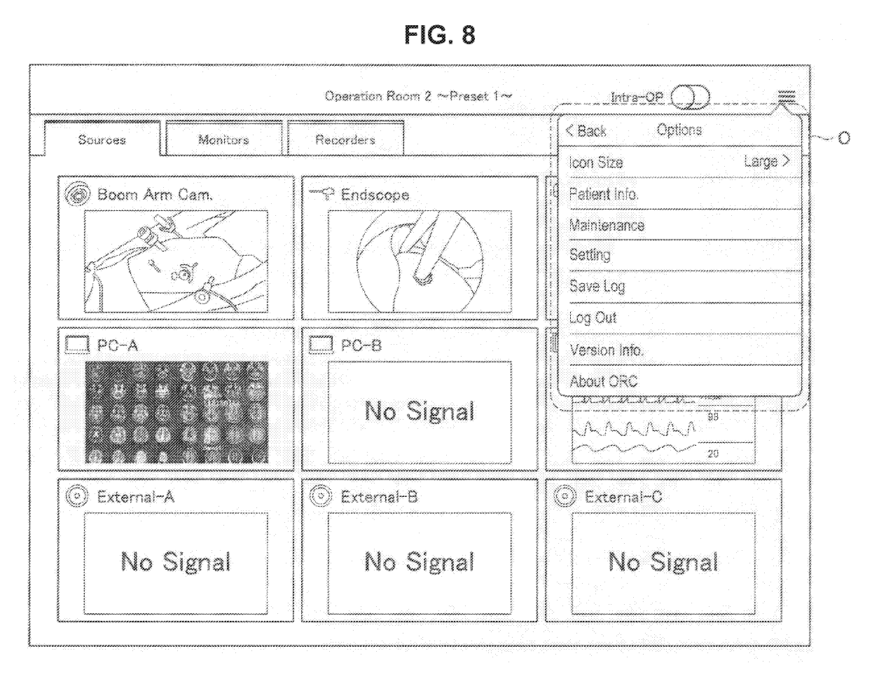

FIG. 8 illustrates an example of the context menu displayed in the case where the context menu button is manipulated. In the case where the context menu button is manipulated, as indicated in O in FIG. 8, context menu associated with various kinds of manipulation and editing of data is displayed.

Further, in the case where the Intra-OP switch is put into an ON state which indicates that an operation is being performed, for example, manipulation with respect to the context menu is restricted (an example of restriction of functions by the function control processing according to the present embodiment). Examples of a method for restricting manipulation with respect to the context menu can include, for example, “making items to be restricted in the context menu grayed out so that manipulation cannot be performed”, “disabling manipulation with respect to the context menu” (in this case, for example, the context menu itself is not displayed), or the like.

In the case where “Sources” is selected in the device category selection tab, the device selection screen, for example, as illustrated in FIG. 2 is displayed.

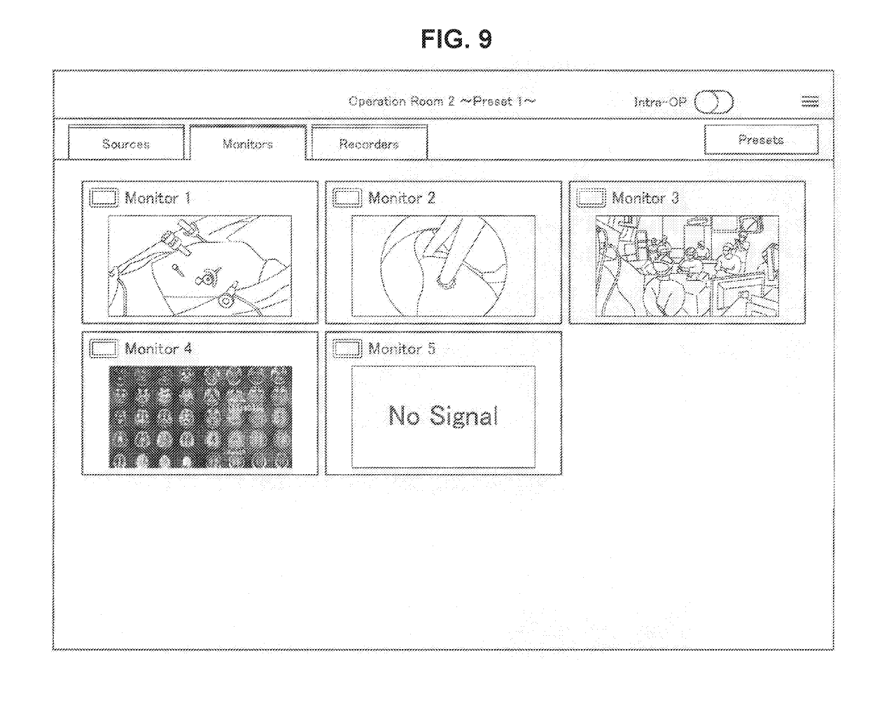

FIG. 9 illustrates another example of the device selection screen and illustrates a case where “Monitors” is selected in the device category selection tab.

Also in the case where “Monitors” is selected in the device category selection tab, the device selection screen has a configuration similar to that in the case where “Sources” is selected illustrated in FIG. 2.

Here, the device selection screen illustrated in FIG. 9 is different from the device selection screen illustrated in FIG. 2 in that “the screen transitions to a Monitors screen in the device control screen which will be described later by the device selection button being manipulated”.

Further, while not illustrated, also in the case where “Recorders” is selected in the device category selection tab, the device selection screen has a configuration similar to that in the case where “Sources” is selected illustrated in FIG. 2.

Here, the device selection screen in the case where “Recorders” is selected in the device category selection tab is different from the device selection screen illustrated in FIG. 2 in that “the screen transitions to a Recorders screen in the device control screen which will be described later by the device selection button being manipulated”.

### (II) Device Control Screen

The device control screen will be described next.

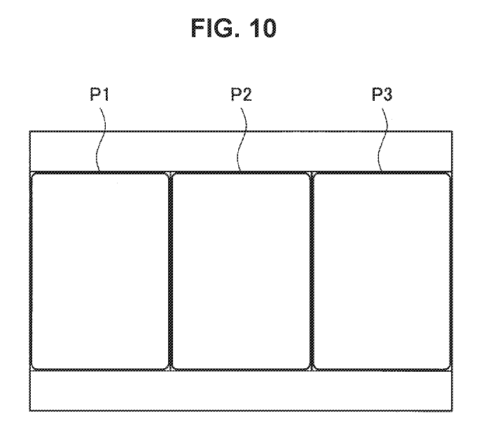

FIG. 10 is an explanatory diagram for explaining an example of display in the medical control system 1000 to which the control method according to the present embodiment is applied and illustrates an example of a configuration of the device control screen displayed on the display screen of the display target apparatus 500.

The device control screen is configured with, for example, the following three panes (display regions).
Device pane (hereinafter, indicated with reference numeral P1)
Control pane (hereinafter, indicated with reference numeral P2)
Connect pane (hereinafter, indicated with reference numeral P3)

Here, positions where the three panes are to be displayed change in accordance with, for example, types of the device control screen (for example, a Sources screen, a Monitors screen and a Recorders screen which will be described later).

Each of the Sources screen, the Monitors screen and the Recorders screen will be described below as an example of the device control screen.

### (II-1) First Example of Device Control Screen: Sources Screen

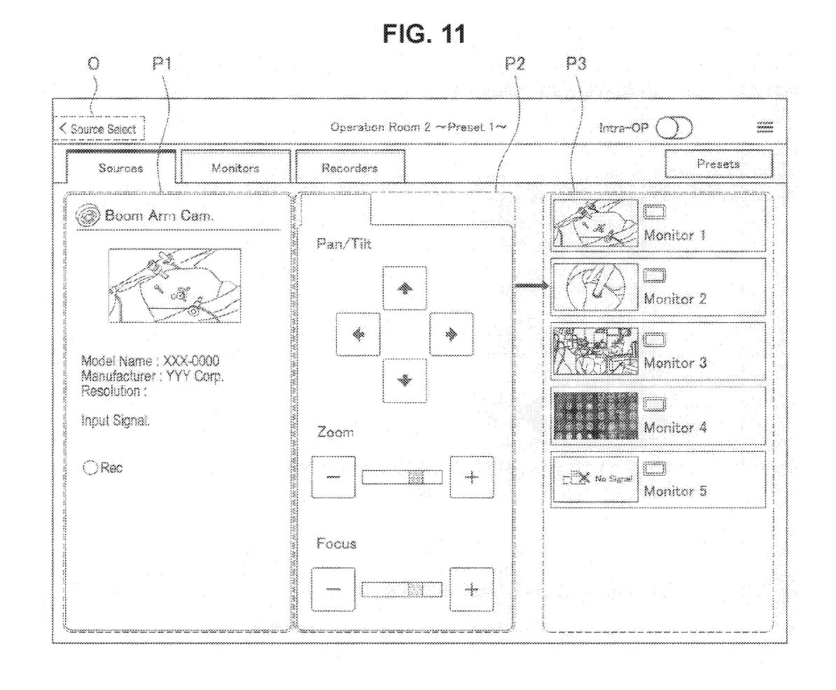

FIG. 11 to FIG. 16 are explanatory diagrams for explaining an example of display in the medical control system 1000 to which the control method according to the present embodiment is applied. FIG. 11 to FIG. 16 each illustrates the whole of the device control screen or part of the device control screen displayed on the display screen of the display target apparatus 500.

The first example of the device control screen will be described below while referring to FIG. 11 to FIG. 16 as appropriate.

FIG. 11 illustrates an example of the Sources screen in the device control screen.

In the Sources screen illustrated in FIG. 11, for example, a button (O illustrated in FIG. 11) for making the screen transition to the device selection screen in which “Sources” is selected in the device category selection tab is provided. The user can select another device again in the device selection screen by manipulating the above-described button (O illustrated in FIG. 11).

Further, the Sources screen illustrated in FIG. 11 includes the Device pane P1, the Control pane P2 and the Connect pane P3 from the left side in the display screen.

In the Device pane P1 illustrated in FIG. 11, information of the device corresponding to the device selection button (O3 illustrated in FIG. 2) selected in the device selection screen in which “Sources” is selected in the device category selection tab is displayed.

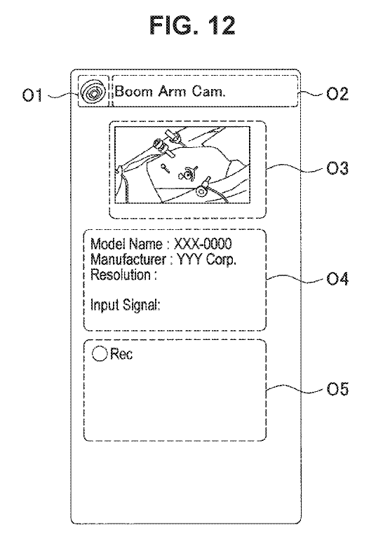

FIG. 12 illustrates an example of a configuration of the Device pane P1. The Device pane P1 includes, for example, an icon (O1 illustrated in FIG. 12), device name display (O2 illustrated in FIG. 12), a thumbnail image (O3 illustrated in FIG. 12), property display (O4 illustrated in FIG. 12) and status display (O5 illustrated in FIG. 12).

The icon (O1 illustrated in FIG. 12) is an icon indicating a type of the device.

The device name display (O2 illustrated in FIG. 12) indicates name of the device. A region of the device name display (O2 illustrated in FIG. 12) is set so as to stretch over the width of the Device pane P1 so that, for example, long device name can be displayed.

The thumbnail image (O3 illustrated in FIG. 12) indicates an image output from a device which is an input source.

Here, while examples of the thumbnail image can include a still image, the thumbnail image may be a moving image. As described above, the thumbnail image according to the present embodiment can be regularly or irregularly updated. In the case where the thumbnail image is updated, it is possible to reflect an image most recently output from the input source in the thumbnail image.

Further, for example, in the case where a parameter of the device which is the input source is adjusted, or the like, in the Control pane P2 of the device control screen, an image output from the input source after the parameter is adjusted is reflected in the thumbnail image.

Further, in the case where a thumbnail image cannot be acquired, display as illustrated in B of FIG. 5 or C of FIG. 5 may be performed in the region for the thumbnail image.

The property display (O4 illustrated in FIG. 12) indicates, for example, information acquired from the device. FIG. 12 illustrates an example where model name and a manufacturer are displayed.

The status display (O5 illustrated in FIG. 12) indicates a state of the device, such as, for example, error display and an operation state.

Referring to FIG. 11 again, an example of the Sources screen in the device control screen will be described. In the Control pane P2 illustrated in FIG. 11, objects for controlling the device indicated in the Device pane P1 are displayed.

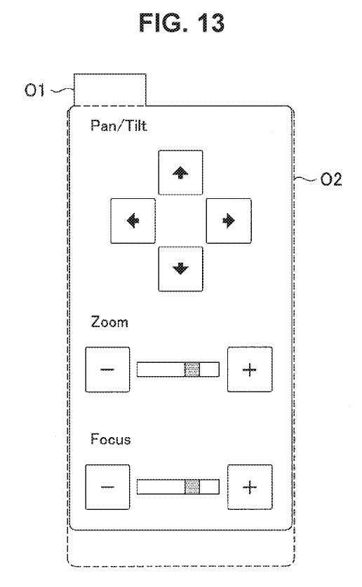

FIG. 13 illustrates an example of a configuration of the Control pane P2. The Control pane P2 includes, for example, a Control tab (O1 illustrated in FIG. 13) and a parameter editing region (O2 illustrated in FIG. 13).

The Control tab (O1 illustrated in FIG. 13) is a tab for each category of control. While FIG. 11 illustrates an example where one Control tab is provided in the Control pane P2, a plurality of Control tabs may be provided.

In the parameter editing region (O2 illustrated in FIG. 13), an object for manipulating the device indicated in the Device pane P1 and an object for adjusting a parameter of the device indicated in the Device pane P1 are displayed. In the parameter editing region (O2 illustrated in FIG. 13) illustrated in FIG. 13, an example is illustrated where a button for performing pan operation and tilt operation, an adjustment button and an adjustment slide bar for adjusting a zoom amount and an adjustment button and an adjustment slide bar for adjusting a focus amount are provided.

Note that, in the case where the whole objects cannot be displayed within the parameter editing region (O2 illustrated in FIG. 13), it may be possible to scroll display the objects. Further, a current zoom amount, a current focus amount, or the like, may be displayed with numerical values in the parameter editing region (O2 illustrated in FIG. 13). Further, items to be displayed in the parameter editing region (O2 illustrated in FIG. 13) can differ in accordance with a type of the device indicated in the Device pane P1.

Referring to FIG. 11 again, an example of the Sources screen in the device control screen will be described. In the Connect pane P3 illustrated in FIG. 11, objects relating to the output destination apparatus 300, at which an image output from the device indicated in the Device pane P1 can be displayed are displayed.

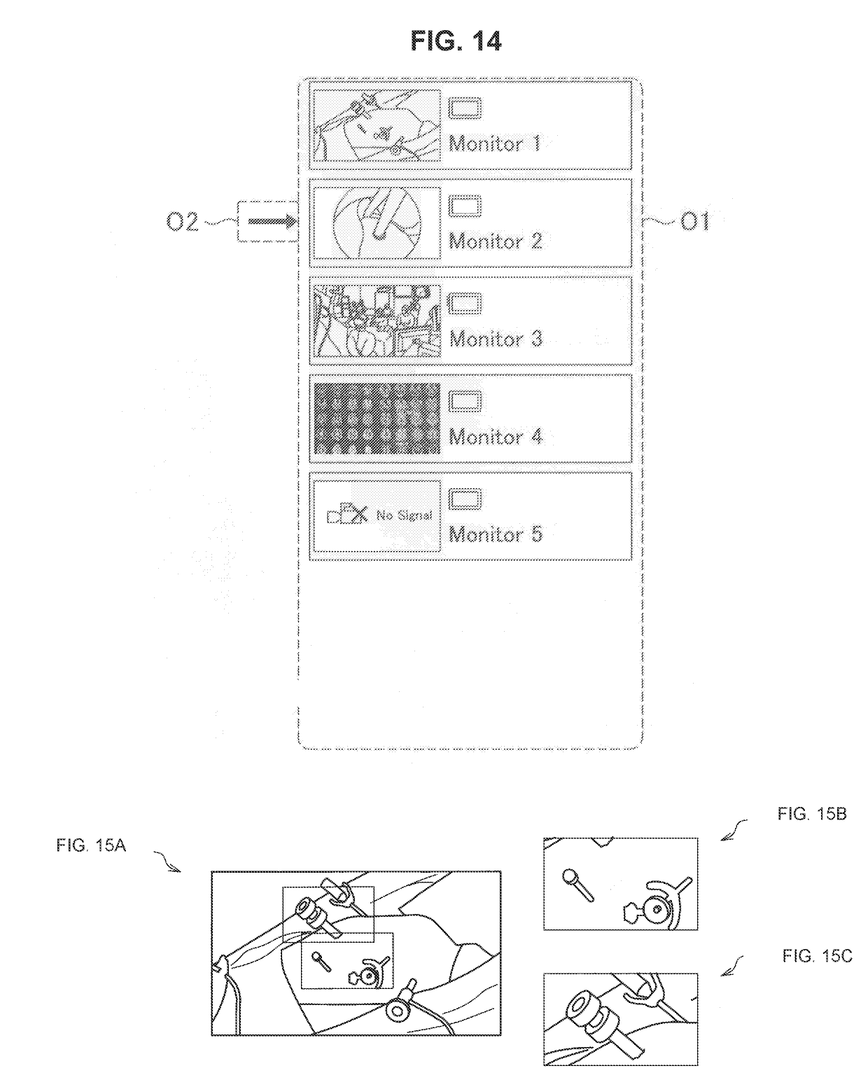

FIG. 14 illustrates an example of a configuration of the Connect pane P3. The Connect pane P3 includes, for example, display of a list of connection destinations (O1 illustrated in FIG. 14) and connection display (O2 illustrated in FIG. 14).

The display of a list of connection destinations (O1 illustrated in FIG. 14) indicates a list of output destination apparatuses 300 at which an image output from the device indicated in the Device pane P1 can be displayed. Examples of the output destination apparatuses 300 displayed in the display of a list of connection destinations (O1 illustrated in FIG. 14) can include, for example, the output destination apparatus 300 for which normal connection with the device indicated in the Device pane P1 is ensured upon construction (or updating) of the medical control system 1000.

Further, the display of a list of connection destinations (O1 illustrated in FIG. 14) includes, for example, a “thumbnail image corresponding to the image displayed at the output destination apparatus 300”, an “icon indicating the output destination apparatus 300” and “device name display of the output destination apparatus 300”.

Here, while examples of the thumbnail image can include a still image, the thumbnail image may be a moving image. As described above, the thumbnail image according to the present embodiment can be regularly or irregularly updated. In the case where the thumbnail image is updated, it is possible to reflect an image most recently input to the output destination apparatus 300 in the thumbnail image. Further, for example, in the case where a parameter of the input source apparatus 200 is adjusted, or the like, in the Control pane P2 of the device control screen illustrated in FIG. 13, in the thumbnail image corresponding to the output destination apparatus 300 at which an image output from the input source apparatus 200 is displayed, an image output from the input source apparatus 200 after the parameter is adjusted is reflected.

Further, in the case where the thumbnail image cannot be acquired, display as illustrated in B of FIG. 5 or C of FIG. 5 may be performed in the region for the thumbnail image.

The connection display (O2 illustrated in FIG. 14) indicates the output destination apparatus 300 selected through user manipulation with respect to the Connect pane P3.

FIG. 14 illustrates an example where the selected output destination apparatus 300 is indicated with an arrow. Note that a method for specifying the selected output destination apparatus 300 is not limited to the example illustrated in FIG. 14. Further, in the case where a plurality of output destination apparatuses 300 are selected through user manipulation with respect to the Connect pane P3, a plurality of types of connection display are displayed in the Connect pane P3.

Here, example of the user manipulation with respect to the Connect pane P3 can include, for example, the following examples. Note that, it goes without saying that the examples of the user manipulation with respect to the Connect pane P3 are not limited to the following examples.
Manipulation of touching the Connect pane P3 (in the case where a display device of the display target apparatus 500 is a touch panel)
Manipulation using a manipulation device such as a physical button, provided at the display target apparatus 500 or an external manipulation device such as a remote controller

Note that the Sources screen in the device control screen is not limited to the example illustrated in FIG. 11.

FIG. 15 illustrates an example of an image to be output from the device indicated in the Device pane P1.

There can be a case where an image to be output from the device indicated in the Device pane P1 includes, for example, an image with higher resolution than that of an image with 2K resolution or an image with high definition (HD) resolution, such as an image with 4K resolution and an image with 8K resolution.

Here, in the case where the image to be output from the device indicated in the Device pane P1 is an image with high resolution as described above, it is assumed that “a user such as a healthcare professional desires to display an image corresponding to part of the image illustrated in A of FIG. 15, for example, as illustrated in B of FIG. 15 and C of FIG. 15 at a different output destination apparatus 300”.

Therefore, the Sources screen in the device control screen may be a screen which enables an image corresponding to part of the image to be displayed at a different output destination apparatus 300 on the basis of the user manipulation. By causing an image corresponding to part of the image to be displayed at a different output destination apparatus 300, for example, it is possible to “cause an image obtained by changing an angle of view of the image of one input source apparatus 200 to be displayed at a different output destination apparatus 300”.

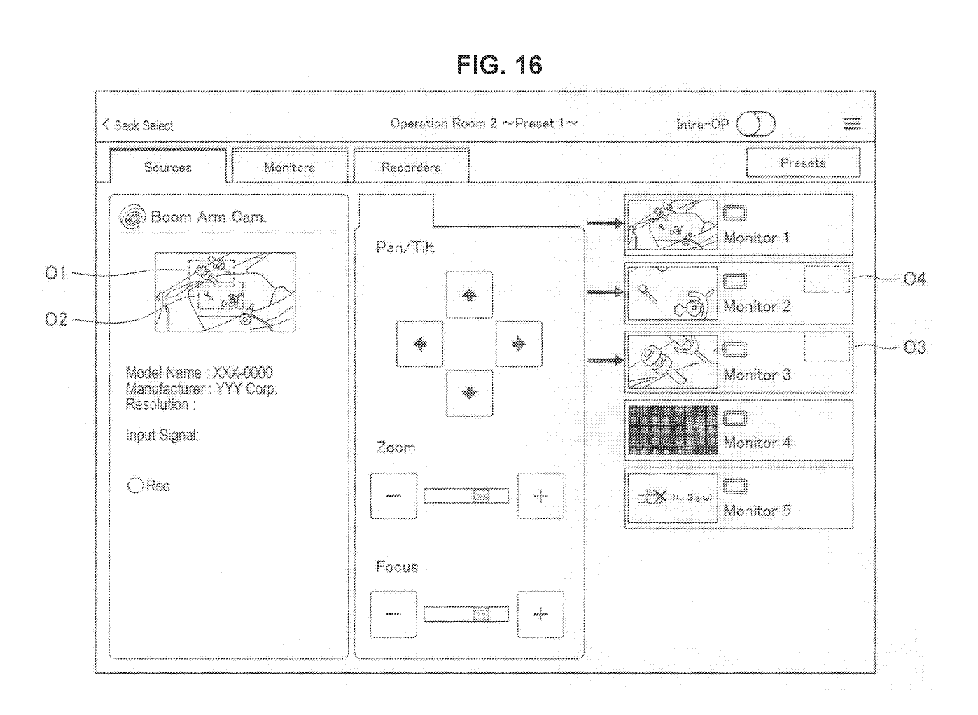

FIG. 16 illustrates another example of the Sources screen in the device control screen.

While the configuration of the Sources screen illustrated in FIG. 16 is basically similar to the configuration of the Sources screen illustrated in FIG. 11, there is a difference in that “the output destination apparatus 300 at which an image corresponding to part of the image output from the device indicated in the Device pane P1 is to be displayed is specified”.

For example, in the case where part of the thumbnail image is selected as illustrated in O1 and O2 in FIG. 16 through user manipulation with respect to the Device pane P1 and the output destination apparatus 300 is selected through user manipulation with respect to the Connect pane P3, as illustrated in O3 and O4 in FIG. 16, the output destination apparatus 300 at which an image of the selected part of the thumbnail image is to be displayed is further specified.

Here, examples of the user manipulation with respect to the Device pane P1 can include, for example, the following examples. Note that, it goes without saying that the examples of the user manipulation with respect to the Device pane P1 are not limited to the following examples.
Manipulation which can select a region on a display screen, such as pinch manipulation with respect to a thumbnail image of the Device pane P1 (in the case where the display device of the display target apparatus 500 is a touch panel)
Manipulation using a manipulation device such as a physical button, provided at the display target apparatus 500 or an external manipulation device such as a remote controller

### (II-2) Second Example of Device Control Screen: Monitors Screen

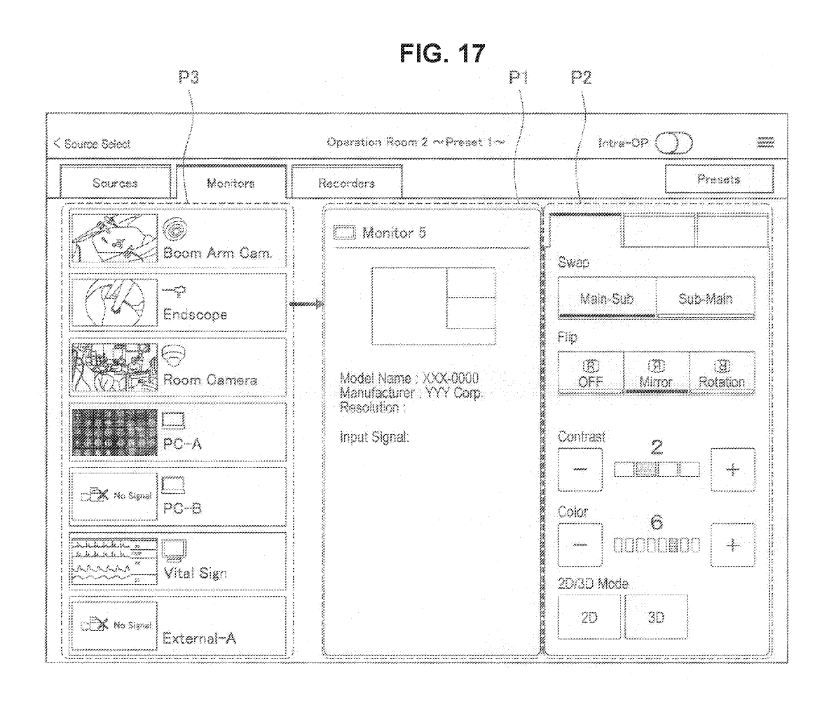

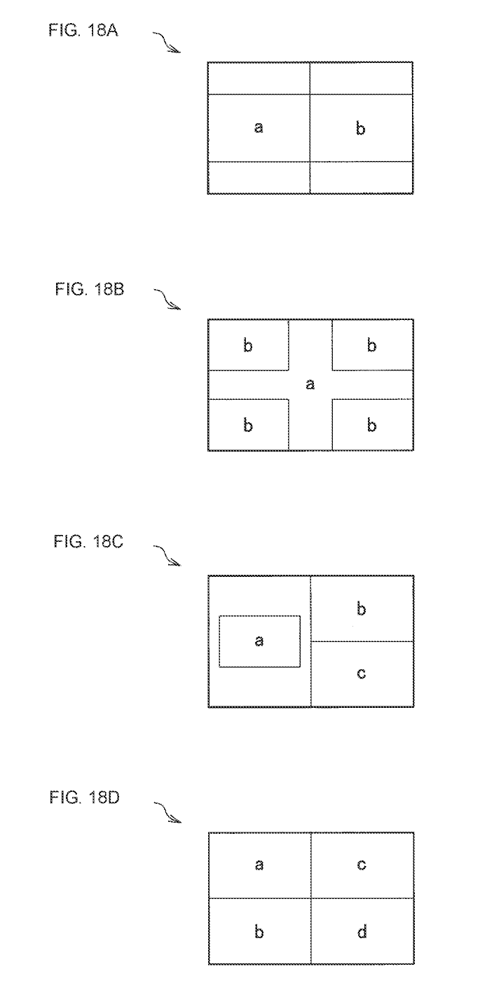

FIG. 17 and FIG. 18 are explanatory diagrams for explaining an example of display in the medical control system 1000 to which the control method according to the present embodiment is applied. FIG. 17 and FIG. 18 each illustrates the whole of the device control screen or part of the device control screen displayed on the display screen of the display target apparatus 500.

A second example of the device control screen will be described below while referring to FIG. 17 and FIG. 18 as appropriate.

FIG. 17 illustrates an example of the Monitors screen in the device control screen.

The Monitors screen illustrated in FIG. 17 includes the Connect pane P3, the Device pane P1 and the Control pane P2 from the left side of the display screen.

In the Connect pane P3 illustrated in FIG. 17, objects relating to the input source apparatus 200, which can be displayed at the output destination apparatus 300 selected in the device selection screen in which “Monitors” is selected in the device category selection tab, are displayed.

FIG. 17 illustrates an example where a list of objects respectively relating to the input source apparatuses 200 is displayed in the Connect pane P3. Examples of the objects relating to the input source apparatuses 200 can include, for example, a thumbnail image, an icon and device name display as with the case of the display of a list of connection destinations (O1 illustrated in FIG. 14) illustrated in FIG. 14.

Further, examples of the input source apparatus 200 displayed in the Connect pane P3 can include, for example, the input source apparatus 200 for which normal connection with the device indicated in the Device pane P1 is ensured upon construction (or updating) of the medical control system 1000.

In the Device pane P1 illustrated in FIG. 17, information of the device corresponding to the device selection button selected in the device selection screen in which “Monitors” is selected in the device category selection tab is displayed. Here, the configuration of the Device pane P1 illustrated in FIG. 17 is similar to the configuration of the Device pane P1, for example, illustrated in FIG. 12.

In the Control pane P2 illustrated in FIG. 17, objects for controlling the device indicated in the Device pane P1 are displayed.

Examples of the objects for controlling the device indicated in the Device pane P1, displayed in the Control pane P2 of the Monitors screen in the device control screen can include the following examples.
Button indicated as “Swap” in FIG. 17: a button for switching between a main display region and a sub display region in the case where the device indicated in the Device pane P1 has a PinP function (function of allowing a plurality of display regions in which images can be independently displayed to be provided in one screen)
Button indicated as “Flip” in FIG. 17: a button for controlling a direction of display in the device indicated in the Device pane P1
Adjustment button indicated as “Contrast” in FIG. 17: a button for adjusting contrast in the device indicated in the Device pane P1
Adjustment button indicated as “Color” in FIG. 17: a button for adjusting luminance, color phase or balance of RGB in the device indicated in the Device pane P1
Button indicated as “2D/3D Mode” in FIG. 17: a button for controlling switching of a display mode associated with stereoscopic view in the device indicated in the Device pane P1

Note that the objects for controlling the device indicated in the Device pane P1, displayed in the Control pane P2 of the Monitors screen in the device control screen are not limited to the above-described examples.

For example, in the case where the device indicated in the Device pane P1 has a PinP function, in the Control pane P2 of the Monitors screen in the device control screen, an object for changing a configuration of the display region in the PinP function may be further displayed. Note that the object for changing the configuration of the display region in the PinP function can be also displayed in other panes such as the Device pane P1.

Examples of the configuration of the display region in the PinP function can include, for example, examples illustrated in A of FIG. 18 to D of FIG. 18. In A of FIG. 18 to D of FIG. 18, the display regions indicated with “a”, “b”, “c” and “d” correspond to the display regions in the PinP function.

Further, an object for controlling the device indicated in the Device pane P1, displayed in the Control pane P2 of the Monitors screen in the device control screen can differ in accordance with a type of the device indicated in the Device pane P1.

For example, in the case where the device indicated in the Device pane P1 does not have a PinP function, the button indicated as “Flip” in FIG. 17 does not have to be displayed.

### (II-3) Third Example of Device Control Screen: Recorders Screen

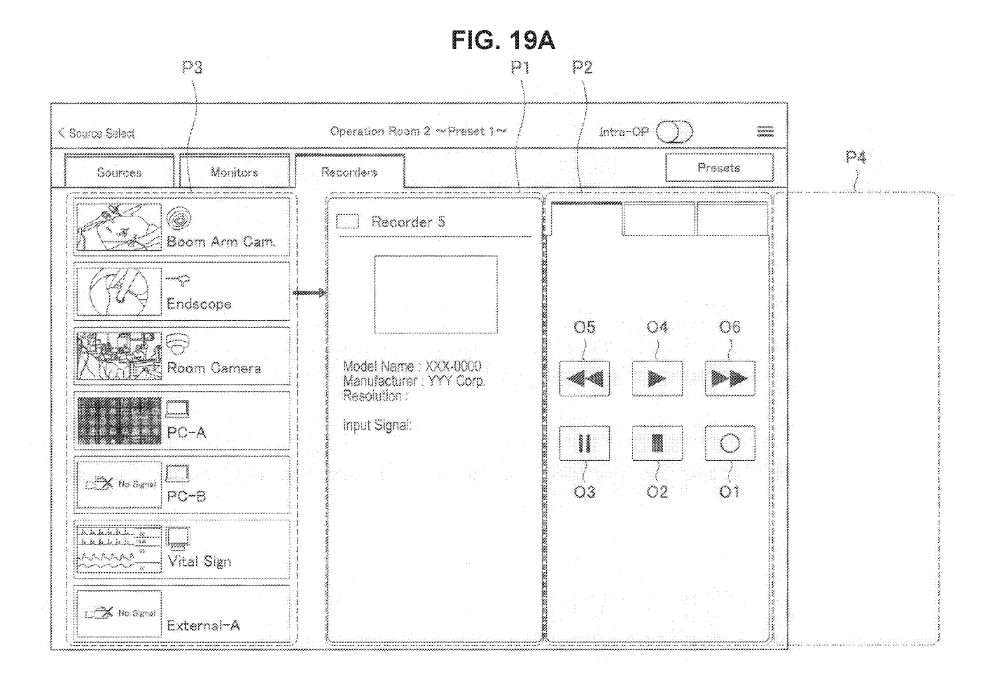

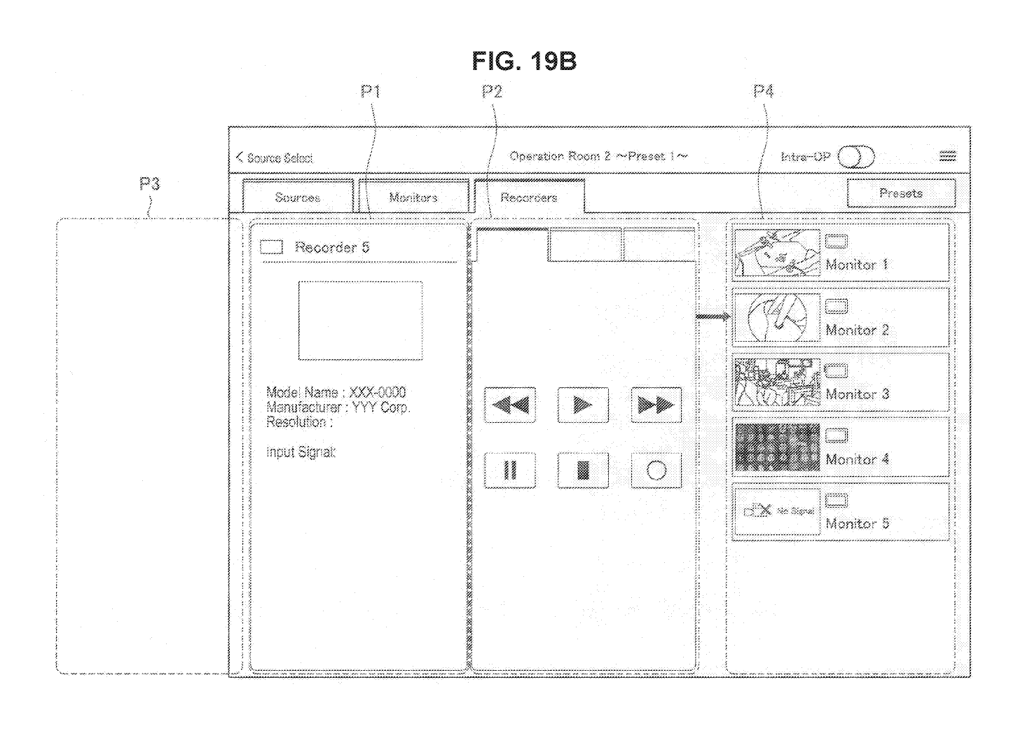

FIG. 19A and FIG. 19B are explanatory diagrams for explaining an example of display in the medical control system 1000 to which the control method according to the present embodiment is applied. FIG. 19A and FIG. 19B each illustrates the whole of the device control screen displayed on the display screen of the display target apparatus 500. The screen is switched between the device control screen illustrated in FIG. 19A and the device control screen illustrated in FIG. 19B through user manipulation.

A third example of the device control screen will be described below while referring to FIG. 19A and FIG. 19B as appropriate.

FIG. 19A illustrates an example of the Recorders screen in the device control screen.

The Recorders screen illustrated in FIG.19A includes the Connect pane P3, the Device pane P1 and the Control pane P2 from the left side of the display screen in a similar manner to the Monitors screen illustrated in FIG. 17.

In the Connect pane P3 illustrated in FIG. 19A, objects relating to the input source apparatus 200 which can output an image signal to the apparatus 400 selected in the device selection screen in which “Recorders” is selected in the device category selection tab are displayed.

FIG. 19A illustrates an example where a list of objects each relating to the input source apparatus 200 is displayed in the Connect pane P3 as with the case of FIG. 17. Further, examples of the input source apparatus 200 displayed in the Connect pane P3 illustrated in FIG. 19A can include, for example, the input source apparatus 200 for which normal connection with the device indicated in the Device pane P1 is ensured upon construction (or updating) of the medical control system 1000.

In the Device pane P1 illustrated in FIG. 19A, information of the device corresponding to the device selection button selected in the device selection screen in which “Recorders” is selected in the device category selection tab is displayed. Here, the configuration of the Device pane P1 illustrated in FIG. 19A is similar to the configuration of the Device pane P1, for example, illustrated in FIG. 12.

In the Control pane P2 illustrated in FIG. 19A, objects for controlling the device indicated in the Device pane P1 are displayed.

Examples of the objects for controlling the device indicated in the Device pane P1, which are displayed in the Control pane P2 of the Recorders screen in the device control screen can include the following examples. Note that, it goes without saying that the examples of the objects for controlling the device indicated in the Device pane P1, which are displayed in the Control pane P2 of the Recorders screen in the device control screen are not limited to the following examples.
Recording button for starting recording of image data in a recording medium (O1 illustrated in FIG. 19A)
Stop button for stopping processing being performed (O2 illustrated in FIG. 19A)
Pause button for pausing processing being performed (O3 illustrated in FIG. 19A)
Reproduction button for starting reproduction of image data stored in a recording medium (O4 illustrated in FIG. 19A)
Rewind button for rewinding an image being reproduced (O5 illustrated in FIG. 19A)
Fast-forward button for fast-forwarding an image being reproduced (O6 illustrated in FIG. 19A)

According to the device control screen illustrated in FIG. 19A, it is, for example, possible to control recording of image data in the device indicated in the Device pane P1 from the input source apparatus 200 selected through user manipulation with respect to the Connect pane P3.

FIG. 19B illustrates another example of the Recorders screen in the device control screen.

The Recorders screen illustrated in FIG. 19B includes the Device pane P1, the Control pane P2 and the Connect pane P4 from the left side of the display screen.

The Device pane P1 illustrated in FIG. 19B and the Control pane P2 illustrated in FIG. 19B are respectively the same as the Device pane P1 illustrated in FIG. 19A and the Control pane P2 illustrated in FIG. 19A.

In the Connect pane P4 illustrated in FIG. 19B, objects relating to the output destination apparatus 300 at which an image output from the device indicated in the Device pane P1 can be displayed are displayed as with the case of the Connect pane P3 illustrated in FIG. 11 and FIG. 14.

According to the device control screen illustrated in FIG. 19B, it is, for example, possible to cause the image reproduced in the device indicated in the Device pane P1 to be displayed at the output destination apparatus 300 selected through user manipulation with respect to the Connect pane P4.

Here, among the apparatuses 400 selected in the device selection screen in which “Recorders” is selected in the device category selection tab, there can exist an apparatus having both an image recording function and an image reproducing function, that is, an apparatus which functions as the output destination and the input source.

In order to control the apparatus 400 which functions as the output destination and the input source as described above, one of the device control screen illustrated in FIG. 19A and the device control screen illustrated in FIG. 19B is insufficient.

Here, for example, if the Connect pane P4 illustrated in FIG. 19B is further displayed on the device control screen illustrated in FIG. 19A, it is possible to sufficiently control the apparatus 400 which functions as the output destination and the input source as described above. However, in the case where a size or resolution of the display screen is not large enough, such as in the case where the display target apparatus 500 is a tablet type apparatus, there is a possibility that visibility degrades by the Connect pane P4 illustrated in FIG. 19B being further displayed in the device control screen illustrated in FIG. 19A.

Therefore, the medical control apparatus 100 causes the screen to be switched between, for example, the device control screen illustrated in FIG. 19A and the device control screen illustrated in FIG. 19B and displayed on the basis of user manipulation.

Here, examples of the user manipulation for switching display of the device control screen can include, for example, the following examples. Note that, it goes without saying that the examples of the user manipulation for switching the display of the device control screen are not limited to the following examples.
Manipulation which can switch the display of the display screen, such as manipulation of swiping the display screen (in the case where the display device of the display target apparatus 500 is a touch panel)
Manipulation using a manipulation device such as a physical button provided at the display target apparatus 500 or an external manipulation device such as a remote controller

By the medical control apparatus 100 causing the screen to be switched between the device control screen illustrated in FIG. 19A and the device control screen illustrated in FIG. 19B and displayed, it becomes possible to prevent degradation of visibility and sufficiently control the apparatus 400 which functions as the output destination and the input source as described above.

### [3] Example of Effects Provided by Control Method According to Present Embodiment Being Used

By the medical control apparatus 100 performing the control processing associated with the control method according to the present embodiment, for example, the following effects are provided. Note that, it goes without saying that the effects provided by the control method according to the present embodiment being used are not limited to the following effects.
For example, it is possible to make it impossible to perform unnecessary manipulation during an operation by a button for switching between a state where an operation is being performed and a state where an operation is not being performed as the Intra-OP switch (for example, O5 illustrated in FIG. 2) displayed in the device selection screen illustrated in FIG. 2, or the like, or the Intra-OP switch displayed in the device control screen illustrated in FIG. 11, or the like, being provided on a user interface (UI).
It is possible to selectively restrict manipulation of the Intra-OP switch in accordance with user authority or authority of the control target apparatus.
It is possible to change functions to be restricted in accordance with user authority or authority of the control target apparatus. Therefore, it is, for example, possible to “even during an operation, at an apparatus to which a specific user is logging in or at a specific apparatus such as an administration terminal, edit matters restricted at other apparatuses”.

### (Medical Control Apparatus According to Present Embodiment)

An example of a configuration of the medical control apparatus according to the present embodiment which can perform the above-described processing associated with the control method according to the present embodiment will be described next.

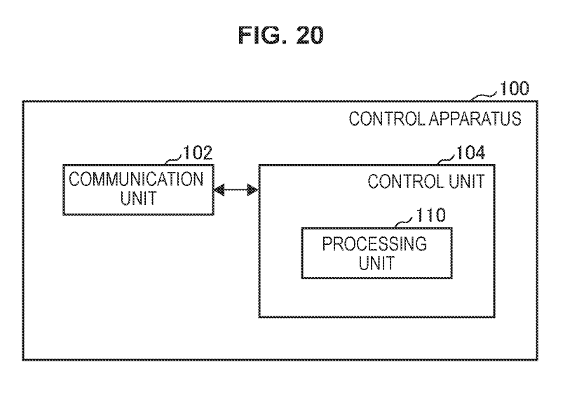

FIG. 20 is a block diagram illustrating an example of the configuration of the medical control apparatus 100 according to the present embodiment. The medical control apparatus 100 includes, for example, a communication unit 102 and a control unit 104.

Further, the medical control apparatus 100 may include, for example, a read only memory (ROM, not illustrated), a random access memory (RAM, not illustrated), a storage unit (not illustrated), an operation unit (not illustrated) which can be manipulated by the user, a display unit (not illustrated) which displays various screens on the display screen, or the like. The medical control apparatus 100, for example, connects the above-described components with a bus which is a data transmission path.

The ROM (not illustrated) stores control data such as a program and an operation parameter to be used by the control unit 104. The RAM (not illustrated) temporarily stores a program to be executed by the control unit 104.

The storage unit (not illustrated) stores, for example, various kinds of data such as data associated with the control method according to the present embodiment, such as user authority information and apparatus authority information, and application. Here, examples of the storage unit (not illustrated) can include, for example, a recording medium such as a non-volatile memory. Further, the storage unit (not illustrated) may be, for example, detachable from the medical control apparatus 100.

Examples of the operation unit (not illustrated) can include, for example, an operation input device which will be described later. Further, examples of the display unit (not illustrated) can include a display device which will be described later.

### [Hardware Configuration Example of Medical Control Apparatus 100]

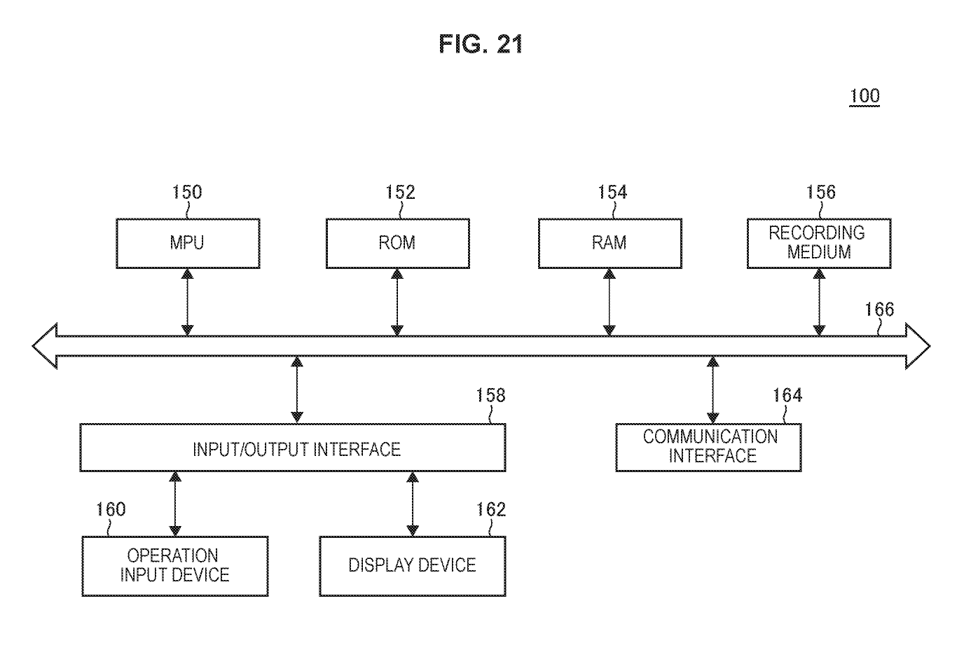

FIG. 21 is an explanatory diagram illustrating an example of a hardware configuration of the medical control apparatus 100 according to the present embodiment. The medical control apparatus 100 includes, for example, an MPU 150, a ROM 152, a RAM 154, a recording medium 156, an input/output interface 158, an operation input device 160, a display device 162 and a communication interface 164. Further, the medical control apparatus 100, for example, connects respective components with a bus 166 which is a data transmission path.

The MPU 150 is configured with, for example, one or more processors configured with an arithmetic circuit such as an MPU, various kinds of processing circuits, or the like, and functions as a control unit (not illustrated) which controls the whole medical control apparatus 100. Further, the MPU 150 plays a role of, for example, a processing unit 110 in the medical control apparatus 100. Note that the processing unit 110 may be configured with a dedicated (or general-purpose) circuit (such as, for example, a processor separate from the MPU 150) which can implement processing at the processing unit 110.

The ROM 152 stores control data such as a program and an operation parameter to be used by the MPU 150. The RAM 154 temporarily stores a program, or the like, to be executed by the MPU 150.

The recording medium 156, which functions as a storage unit (not illustrated), for example, stores data associated with the control method according to the present embodiment, such as user authority information and apparatus authority information, and various kinds of data such as various kinds of application. Here, examples of the recording medium 156 can include, for example, a magnetic recording medium such as a hard disk, and a non-volatile memory such as a flash memory. Further, the recording medium 156 may be detachable from the medical control apparatus 100.

The input/output interface 158, for example, connects the operation input device 160 and the display device 162. The operation input device 160 functions as an operation unit (not illustrated), and the display device 162 functions as a display unit (not illustrated). Here, examples of the input/output interface 158 can include, for example, a universal serial bus (USB) terminal, a digital visual interface (DVI) terminal, a high-definition multimedia interface (HDMI) (registered trademark) terminal, various kinds of processing circuits, or the like.

Further, the operation input device 160 is, for example, provided on the medical control apparatus 100 and is connected to the input/output interface 158 inside the medical control apparatus 100. Examples of the operation input device 160 can include, for example, a button, a direction key, a rotary selector such as a jog dial, combination thereof, or the like.

Further, the display device 162 is, for example, provided on the medical control apparatus 100 and is connected to the input/output interface 158 inside the medical control apparatus 100. Examples of the display device 162 can include, for example, a liquid crystal display, an organic electro-luminescence (EL) display, an organic light emitting diode (OLED) display, or the like.

Note that, it goes without saying that the input/output interface 158 can be connected to an external device such as an external operation input device (such as, for example, a keyboard and a mouse) and an external display device of the medical control apparatus 100. Further, the display device 162 may be a device such as, for example, a touch panel, which can perform display and allows user manipulation.

The communication interface 164 is communication means provided at the medical control apparatus 100. The communication interface 164, for example, functions as a communication unit (not illustrated) for performing communication in a wireless or wired manner with each of one or more external apparatuses constituting the medical control system 1000, such as the input source apparatus 200, the output destination apparatus 300, the apparatus 400, the display target apparatus 500 and other apparatuses 600A and 600B via a network (or directly). Further, the communication interface 164 may, for example, play a role of performing communication in a wireless or wired manner with an external apparatus other than apparatuses constituting the medical control system 1000, such as a server.

Here, examples of the communication interface 164 can include, for example, a communication antenna and a radio frequency (RF) circuit (wireless communication), an IEEE802.15.1 port and a transmission/reception circuit (wireless communication), an IEEE802.11 port and a transmission/reception circuit (wireless communication), a local area network (LAN) terminal and a transmission/reception circuit (wired communication), or the like.

The medical control apparatus 100 performs processing associated with the control method according to the present embodiment, for example, with the configuration illustrated in FIG. 21. Note that the hardware configuration of the medical control apparatus according to the present embodiment is not limited to the configuration illustrated in FIG. 21.

For example, in the case where the medical control apparatus 100 performs communication with external apparatuses, or the like, via a connected external communication device, the communication interface 164 does not have to be provided. Further, the communication interface 164 may be configured to enable communication with one or more external apparatuses using a plurality of communication schemes.

Further, the medical control apparatus 100 can, for example, employ a configuration which does not include one or more of the recording medium 156, the operation input device 160 and the display device 162.

Further, for example, part or the whole of the configuration illustrated in FIG. 21 (or the configuration according to the modified examples) may be implemented with one or more integrated circuits (ICs).

Referring to FIG. 20 again, an example of the configuration of the medical control apparatus 100 will be described. The communication unit 102, which is communication means provided at the medical control apparatus 100, performs communication in a wireless or wired manner with each of one or more external apparatuses constituting the medical control system 1000, such as, for example, the input source apparatus 200, the output destination apparatus 300, the apparatus 400, the display target apparatus 500 and other apparatuses 600A and 600B. Further, the communication unit 102 can also perform communication in a wireless or wired manner with, for example, an external apparatus other than the apparatuses constituting the medical control system 1000, such as a server.

Here, while examples of the communication unit 102 can include, for example, a communication antenna and an RF circuit, a LAN terminal and a transmission/reception circuit, or the like, the configuration of the communication unit 102 is not limited to the above described configuration. For example, the communication unit 102 can employ a configuration supporting arbitrary standards which enable communication, such as a USB terminal and a transmission/reception circuit, or an arbitrary configuration which enables communication with external apparatuses via a network. Further, the communication unit 102 may be configured to enable communication with one or more external apparatuses using a plurality of communication schemes.

The control unit 104, which is configured with, for example, one or more processors configured with an arithmetic circuit such as an MPU, various kinds of processing circuits, or the like, controls the whole of the medical control apparatus 100. Further, the control unit 104 includes, for example, a processing unit 110 and plays a role of leading the processing associated with the control method according to the present embodiment.

The processing unit 110 plays a role of leading the control processing according to the present embodiment. The processing unit 110 restricts functions which can be executed at the control target apparatus in the case where an operation is being performed on the basis of whether or not an operation is being performed. Specifically, the processing unit 110, for example, performs one of the function control processing according to the first example described in the above (1) to the function control processing according to the third example described in the above (3).

Further, the processing unit 110 may further perform one or both of the notification processing according to the first example described in the above (i) and the notification processing according to the second example described in the above (ii).

Further, the processing unit 110 may, for example, perform processing in response to user manipulation performed with respect to the device control screen or the screen such as the device selection screen, displayed at the display target apparatus 500. Examples of the above-described processing in response to the user manipulation can include, for example, “processing of causing the screen to transition to the device control screen corresponding to manipulation in response to the manipulation with respect to the device selection screen”, “processing of connecting the input source apparatus 200 and the output destination apparatus 300 (or the apparatus 400) by controlling various kinds of switches illustrated in FIG. 1 in response to the manipulation with respect to the device control screen”, or the like.

By the control unit 104 including, for example, the processing unit 110, the control unit 104 leads the processing associated with the control method according to the present embodiment.

The medical control apparatus 100 performs the processing (for example, the above-described control processing) according to the control method according to the present embodiment with the configuration illustrated in, for example, FIG. 20.

Therefore, the medical control apparatus 100 can prevent user erroneous manipulation during an operation with the configuration illustrated in, for example, FIG. 20.

Further, the medical control apparatus 100 can provide effects provided by the processing associated with the control method according to the present embodiment being performed, with the configuration illustrated in, for example, FIG. 20.

Further, the medical control system 1000 which can prevent user erroneous manipulation during an operation is realized by the medical control apparatus 100 having the configuration illustrated in, for example, FIG. 20.

Note that the configuration of the medical control apparatus according to the present embodiment is not limited to the configuration illustrated in FIG. 20.

For example, the medical control apparatus according to the present embodiment can include the processing unit 110 illustrated in FIG. 20 separately from the control unit 104 (for example, can implement the processing unit 110 with another processing circuit).

Further, as described above, each of the “above-described function control processing” and the “above-described function control processing and the above-described notification processing” is processing divided from the processing associated with the control method according to the present embodiment for convenience sake. Therefore, the configuration for implementing the processing associated with the control method according to the present embodiment is not limited to the configuration illustrated in FIG. 20 and can be a configuration in accordance with a way of dividing the processing associated with the control method according to the present embodiment.

Further, in the case where communication is performed with an external apparatus via an external communication device having functions and a configuration similar to those of the communication unit 102, the medical control apparatus according to the present embodiment does not have to include the communication unit 102.

While the medical control apparatus has been described above as the present embodiment, the present embodiment is not limited to such an embodiment. The present embodiment can be applied to various kinds of equipment such as, for example, a computer such as a PC, a server and an OR Controller, a tablet type apparatus and a communication apparatus such as a smartphone, which can perform the processing associated with the control method according to the present embodiment. Further, the present embodiment can be applied to, for example, a processing IC which can be incorporated into the equipment as described above.

### (Program According to Present Embodiment)

By a program for causing a computer to function as the medical control apparatus according to the present embodiment (a program which can execute the processing associated with the control method according to the present embodiment such as, for example, the above-described control processing) being executed by a processor, or the like, at the computer, it is possible to prevent user erroneous manipulation during an operation.

Further, by a program for causing a computer to function as the medical control apparatus according to the present embodiment being executed by a processor, or the like, at the computer, it is possible to provide effects provided through the above-described processing associated with the control method according to the present embodiment.

The preferred embodiment of the present disclosure has been described above with reference to the accompanying drawings, whilst the present disclosure is not limited to the above examples. A person skilled in the art may find various alterations and modifications within the scope of the appended claims, and it should be understood that they will naturally come under the technical scope of the present disclosure.

For example, while the configuration where the program (computer program) for causing the computer to function as the medical control apparatus according to the present embodiment is provided has been described above, the present embodiment can also further provide a recording medium in which the above-described program is stored.

The above-described configuration is one example of the present embodiment and naturally falls within the technical scope of the present disclosure.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.
(1)

A medical control apparatus including:

a processing unit configured to restrict a function which is executable at a control target apparatus in a case where an operation is being performed, on a basis of whether or not an operation is being performed.
(2)

The medical control apparatus according to (1),

in which the processing unit causes an object for which a state is switched between a state where an operation is being performed and a state where an operation is not being performed according to user manipulation, and

in a case where the object indicates a state where an operation is being performed, a function which is executable at the control target apparatus is restricted.
(3)

The medical control apparatus according to (2),

in which the processing unit causes the object to be displayed on a display screen on a basis of user authority information corresponding to a user who is utilizing the control target apparatus, and

in a case where the object is not caused to be displayed, a function which is executable at the control target apparatus is not restricted.
(4)

The medical control apparatus according to (2) or (3),
in which the processing unit restricts manipulation with respect to the object on a basis of user authority information corresponding to a user who is utilizing the control target apparatus.
(5)

The medical control apparatus according to (2),

in which the processing unit causes the object to be displayed on a display screen on a basis of apparatus authority information corresponding to the control target apparatus, and

in a case where the object is not caused to be displayed, a function which is executable at the control target apparatus is not restricted.
(6)

The medical control apparatus according to (2) or (5),

in which the processing unit restricts manipulation with respect to the object on a basis of apparatus authority information corresponding to the control target apparatus.
(7)

The medical control apparatus according to any one of (2) to (6),

in which the processing unit notifies a predetermined user that an operation is being performed or an operation is finished on a basis of a state of the object.
(8)

The medical control apparatus according to any one of (1) to (7),

in which the processing unit restricts a function which is executable at the control target apparatus on a basis of user authority information corresponding to a user who is utilizing the control target apparatus.
(9)

The medical control apparatus according to any one of (1) to (7),

in which the processing unit restricts a function which is executable at the control target apparatus on a basis of apparatus authority information corresponding to the control target apparatus.
(10)

The medical control apparatus according to any one of (1) to (9),

in which a function of the control target apparatus to be restricted includes a function of setting information relating to a patient on which an operation is to be performed.
(11)

The medical control apparatus according to any one of (1) to (10),

in which the processing unit causes a notification that a function of the control target apparatus is restricted to be made in a case where the function of the control target apparatus is restricted.
(12)

A control method to be executed by a medical control apparatus, the control method including:

a step of restricting a function which is executable at a control target apparatus in a case where an operation is being performed, on a basis of whether or not an operation is being performed.
(13)

A program for causing a computer to implement a function of restricting a function which is executable at a control target apparatus in a case where an operation is being performed, on a basis of whether or not an operation is being performed.

### REFERENCE SIGNS LIST

100 medical control apparatus
102 communication unit
104 control unit
110 processing unit
200 input source apparatus
300 output destination apparatus
400 apparatus
500 display target apparatus
600 other apparatus
1000 medical control system

1. A medical control apparatus comprising:
a processing unit configured to restrict a function which is executable at a control target apparatus in a case where an operation is being performed, on a basis of whether or not an operation is being performed.

2. The medical control apparatus according to claim 1,
wherein the processing unit causes an object for which a state is switched between a state where an operation is being performed and a state where an operation is not being performed according to user manipulation, and
in a case where the object indicates a state where an operation is being performed, a function which is executable at the control target apparatus is restricted.

3. The medical control apparatus according to claim 2,
wherein the processing unit causes the object to be displayed on a display screen on a basis of user authority information corresponding to a user who is utilizing the control target apparatus, and
in a case where the object is not caused to be displayed, a function which is executable at the control target apparatus is not restricted.

4. The medical control apparatus according to claim 2,
wherein the processing unit restricts manipulation with respect to the object on a basis of user authority information corresponding to a user who is utilizing the control target apparatus.

5. The medical control apparatus according to claim 2,
wherein the processing unit causes the object to be displayed on a display screen on a basis of apparatus authority information corresponding to the control target apparatus, and
in a case where the object is not caused to be displayed, a function which is executable at the control target apparatus is not restricted.

6. The medical control apparatus according to claim 2,
wherein the processing unit restricts manipulation with respect to the object on a basis of apparatus authority information corresponding to the control target apparatus.

7. The medical control apparatus according to claim 2,
wherein the processing unit notifies a predetermined user that an operation is being performed or an operation is finished on a basis of a state of the object.

8. The medical control apparatus according to claim 1,
wherein the processing unit restricts a function which is executable at the control target apparatus on a basis of user authority information corresponding to a user who is utilizing the control target apparatus.

9. The medical control apparatus according to claim 1,
wherein the processing unit restricts a function which is executable at the control target apparatus on a basis of apparatus authority information corresponding to the control target apparatus.

10. The medical control apparatus according to claim 1,
wherein a function of the control target apparatus to be restricted includes a function of setting information relating to a patient on which an operation is to be performed.

11. The medical control apparatus according to claim 1,
wherein the processing unit causes a notification that a function of the control target apparatus is restricted to be made in a case where the function of the control target apparatus is restricted.

12. A control method to be executed by a medical control apparatus, the control method comprising:
a step of restricting a function which is executable at a control target apparatus in a case where an operation is being performed, on a basis of whether or not an operation is being performed.

13. A program for causing a computer to implement a function of restricting a function which is executable at a control target apparatus in a case where an operation is being performed, on a basis of whether or not an operation is being performed.
